(19) United States
(12) Patent Application Publication
(10) Pub. No.: US 2024/0325366 A1
(43) Pub. Date: Oct. 3, 2024

(54) SELECTIVE CDK9 INHIBITOR FOR THE TREATMENT OF A RAS MUTANT CANCER

(71) Applicant: VINCERX PHARMA, INC., Palo Alto, CA (US)

(72) Inventors: Sooin HWANG, San Rafael, CA (US); Beatrix STELTE-LUDWIG, Wülfrath (DE)

(21) Appl. No.: 18/291,834

(22) PCT Filed: Jul. 26, 2022
(86) PCT No.: PCT/US2022/038281

§ 371 Date: Jan. 24, 2024

Related U.S. Application Data
(60) Provisional application No. 63/225,819, filed on Jul. 26, 2021.

(51) Int. Cl. A61K 31/444; A61K 31/4439; A61K 31/52; A61K 45/06; A61P 35/00

(52) U.S. Cl. CPC A61K 31/444; A61K 31/4439; A61K 31/52; A61K 45/06; A61P 35/00

(57) ABSTRACT

The present invention relates generally to the treatment of a cancer that expresses a RAS mutation with a selective CDK9 inhibitor.

### CROSS-REFERENCE

This application claims the benefit of U.S. Provisional Application Ser. No. 63/225,819 filed Jul. 26, 2021 which is hereby incorporated by reference in its entirety.

### BACKGROUND OF THE INVENTION

The family of cyclin-dependent kinase (CDK) proteins consists of members that are key regulators of the cell division cycle (cell cycle CDK's), that are involved in regulation of gene transcription (transcriptional CDK's), and of members with other functions. CDKs require for activation the association with a regulatory cyclin subunit. The cell cycle CDKs CDK1/cyclin B, CDK2/cyclin A, CDK2/cyclinE, CDK4/cyclinD, and CDK6/cyclinD get activated in a sequential order to drive a cell into and through the cell division cycle. The transcriptional CDKs CDK9/cyclin T and CDK7/cyclin H regulate the activity of RNA polymerase II via phosphorylation of the carboxy-terminal domain (CTD). Positive transcription factor b (P-TEFb) is a heterodimer of CDK9 and one of four cyclin partners, cyclin T1, cyclin K, cyclin T2a or T2b.

Whereas CDK9 (NCBI GenBank Gene ID 1025) is exclusively involved in transcriptional regulation, CDK7 in addition participates in cell cycle regulation as CDK-activating kinase (CAK).

Transcription of genes by RNA polymerase II is initiated by assembly of the pre-initiation complex at the promoter region and phosphorylation of Ser 5 and Ser 7 of the CTD by CDK7/cyclin H. For a major fraction of genes RNA polymerase II stops mRNA transcription after it moved 20-40 nucleotides along the DNA template. This promoter-proximal pausing of RNA polymerase II is mediated by negative elongation factors and is recognized as a major control mechanism to regulate expression of rapidly induced genes in response to a variety of stimuli (Cho et al., Cell Cycle 2010, 9, 1697). P-TEFb is crucially involved in overcoming promoter-proximal pausing of RNA polymerase II and transition into a productive elongation state by phosphorylation of Ser 2 of the CTD as well as by phosphorylation and inactivation of negative elongation factors.

Activity of P-TEFb itself is regulated by several mechanisms. About half of cellular P-TEFb exists in an inactive complex with 7SK small nuclear RNA (7SK snRNA), La-related protein 7 (LARP7/PIP7S) and hexamethylene bis-acetamide inducible proteins 1/2 (HEXIM1/2, He et al., Mol. Cell 2008, 29, 588). The remaining half of P-TEFb exists in an active complex containing the bromodomain protein Brd4 (Yang et al., Mol. Cell 2005, 19, 535). Brd4 recruits P-TEFb through interaction with acetylated histones to chromatin areas primed for gene transcription. Through alternately interacting with its positive and negative regulators, P-TEFb is maintained in a functional equilibrium: P-TEFb bound to the 7SK snRNA complex represents a reservoir from which active P-TEFb can be released on demand of cellular transcription and cell proliferation (Zhou & Yik, Microbiol. Mol. Biol. Rev. 2006, 70, 646). Furthermore, the activity of P-TEFb is regulated by posttranslational modifications including phosphorylation/de-phosphorylation, ubiquitination, and acetylation (reviewed in Cho et al., Cell Cycle 2010, 9, 1697).

Deregulated CDK9 kinase activity of the P-TEFb heterodimer is associated with a variety of human pathological settings such as hyper-proliferative diseases (e.g. cancer such as chronic lymphocytic leukemia (CLL)), virally induced infectious diseases or cardiovascular diseases.

Cancer is regarded as a hyper-proliferative disorder mediated by a disbalance of proliferation and cell death (apoptosis). High levels of anti-apoptotic Bcl-2-family proteins are found in various human tumours and account for prolonged survival of tumour cells and therapy resistance. Inhibition of P-TEFb kinase activity was shown to reduce transcriptional activity of RNA polymerase II leading to a decline of short-lived anti-apoptotic proteins, especially Mcl-1 and XIAP, reinstalling the ability of tumour cells to undergo apoptosis. A number of other proteins associated with the transformed tumour phenotype (such as Myc, NF-kB responsive gene transcripts, mitotic kinases) are either short-lived proteins or are encoded by short-lived transcripts which are sensitive to reduced RNA polymerase II activity mediated by P-TEFb inhibition (reviewed in Wang & Fischer, Trends Pharmacol. Sci. 2008, 29, 302).

RAS was identified as an oncogene more than 40 years ago, and it is one of the most mutated genes in human cancer. A recent analysis estimated that about 19% of cancer patients carry a RAS mutation, with 75% of them being KRAS mutations. In particular, KRAS mutations predominate in pancreatic adenocarcinoma (88%), colorectal adenocarcinoma (50%), and lung adenocarcinoma (32%). KRAS mutations are found in three hotspots: codons 12, 13, and 61, amino acids that are within the guanine nucleotide-binding interface. These missense mutations result in increased nucleotide exchange (GDP for GTP) and/or decreased GTP hydrolysis, and consequently hyperactive KRAS. Aberrant downstream signaling activation (e.g., RAF/MEK) leads to uncontrolled cell proliferation and ultimately tumor formation. Given the prevalence of KRAS mutations in human cancers, KRAS has been an attractive target for drug development. Despite significant efforts, directly targeting oncogenic KRAS has proven to be challenging.

### SUMMARY OF THE INVENTION

Disclosed herein is a method of treating RAS mutant cancers in subjects in need thereof with a selective CDK9 inhibitor. In some embodiments, the selective CDK9 inhibitor is well-tolerated with minimal adverse effects in subjects in need thereof.

Also disclosed herein is a method of treating a cancer that expresses a mutant form of a RAS protein in a subject in need thereof, the method comprising administering to the subject in need a therapeutically-effective amount of a selective CDK9 inhibitor. In some embodiments of a method of treating a cancer that expresses a mutant form of a RAS protein, the RAS protein is a mutant form of a NRAS protein. In some embodiments of a method of treating a cancer that expresses a mutant form of a RAS protein, the RAS protein is a mutant form of a HRAS protein. In some embodiments of a method of treating a cancer that expresses a mutant form of a RAS protein, the RAS protein is a mutant form of a KRAS protein. In some embodiments of a method of treating a cancer that expresses a mutant form of a RAS protein, the mutant form of the RAS protein comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V. In some embodiments of a method of treating a cancer that expresses a mutant form of a RAS protein, the mutant form of the RAS protein comprises one or more amino acid substitutions selected from the group consisting of G12C, G12D, G12V, G13S, and Q61R. In some embodiments of a method of treating a cancer that expresses a mutant form of a RAS protein, the mutant form of the RAS protein comprises the amino acid substitution G12S. In some embodiments of a method of treating a cancer that expresses a mutant form of a RAS protein, the mutant form of the RAS protein comprises the amino acid substitution G12C. In some embodiments of a method of treating a cancer that expresses a mutant form of a RAS protein, the mutant form of the RAS protein comprises the amino acid substitution G12D. In some embodiments of a method of treating a cancer that expresses a mutant form of a RAS protein, the mutant form of the RAS protein comprises the amino acid substitution G12V. In some embodiments of a method of treating a cancer that expresses a mutant form of a RAS protein, the mutant form of the RAS protein comprises the amino acid substitution G13S. In some embodiments of a method of treating a cancer that expresses a mutant form of a RAS protein, the mutant form of the RAS protein comprises the amino acid substitution G13D. In some embodiments of a method of treating a cancer that expresses a mutant form of a RAS protein, the mutant form of the RAS protein comprises the amino acid substitution Q61R. In some embodiments of a method of treating a cancer that expresses a mutant form of a RAS protein, the mutant form of the RAS protein comprises the amino acid substitution Q61H. In some embodiments of a method of treating a cancer that expresses a mutant form of a RAS protein, the mutant form of the RAS protein comprises a secondary adaptive RAS mutation. In some embodiments of a method of treating a cancer that expresses a mutant form of a RAS protein, the cancer is bladder cancer, brain cancer, brain metastases, breast cancer, cervical cancer, colorectal cancer, endometrial cancer, gastrointestinal cancer, glioblastoma, head and neck cancer, leukemias, liver cancer, lung cancer, lymphomas, melanoma, multiple myeloma, ovarian cancer, pancreatic cancer, peritoneal cancer, prostate cancer, renal cancer, salivary gland carcinoma, thyroid cancer, or uterine carcinoma. In some embodiments of a method of treating a cancer that expresses a mutant form of a RAS protein, the cancer is non-small cell lung cancer, pancreatic cancer, or glioblastoma. In some embodiments of a method of treating a cancer that expresses a mutant form of a RAS protein, the cancer is colorectal cancer, non-small cell lung cancer, or pancreatic cancer. In some embodiments of a method of treating a cancer that expresses a mutant form of a RAS protein, the cancer is glioblastoma. In some embodiments of a method of treating a cancer that expresses a mutant form of a RAS protein, the cancer is colorectal cancer. In some embodiments of a method of treating a cancer that expresses a mutant form of a RAS protein, the cancer is non-small cell lung cancer. In some embodiments of a method of treating a cancer that expresses a mutant form of a RAS protein, the cancer is pancreatic cancer. In some embodiments of a method of treating a cancer that expresses a mutant form of a RAS protein, the selective CDK9 inhibitor is administered in multiple 21-day cycles. In some embodiments of a method of treating a cancer that expresses a mutant form of a RAS protein, the selective CDK9 inhibitor is administered in at least one 21-day cycle. In some embodiments of a method of treating a cancer that expresses a mutant form of a RAS protein, the selective CDK9 inhibitor is administered in at least two 21-day cycles. In some embodiments of a method of treating a cancer that expresses a mutant form of a RAS protein, the selective CDK9 inhibitor is administered in at least three 21-day cycles. In some embodiments of a method of treating a cancer that expresses a mutant form of a RAS protein, the selective CDK9 inhibitor is administered in at least four 21-day cycles. In some embodiments of a method of treating a cancer that expresses a mutant form of a RAS protein, the selective CDK9 inhibitor is administered in at least five 21-day cycles. In some embodiments of a method of treating a cancer that expresses a mutant form of a RAS protein, the compound is administered in at least six 21-day cycles. In some embodiments of a method of treating a cancer that expresses a mutant form of a RAS protein, the selective CDK9 inhibitor is administered in at least 12 21-day cycles. In some embodiments of a method of treating a cancer that expresses a mutant form of a RAS protein, the selective CDK9 inhibitor is administered once weekly. In some embodiments of a method of treating a cancer that expresses a mutant form of a RAS protein, the selective CDK9 inhibitor is administered twice weekly. In some embodiments of a method of treating a cancer that expresses a mutant form of a RAS protein, the selective CDK9 inhibitor is administered weekly three days on and four days off. In some embodiments of a method of treating a cancer that expresses a mutant form of a RAS protein, the method further comprises administering an additional therapeutic agent. In some embodiments of a method of treating a cancer that expresses a mutant form of a RAS protein, the additional therapeutic agent is an AKT inhibitor, BCL2 inhibitor, BTK inhibitor, EGFR inhibitor, farnesyl transferase inhibitor, HER2 inhibitor, MEK inhibitor, mTOR inhibitor, PI3K inhibitor, RAF inhibitor, RAS inhibitor, or any combinations thereof. In some embodiments of a method of treating a cancer that expresses a mutant form of a RAS protein, the selective CDK9 inhibitor and the additional therapeutic agent are administered sequentially. In some embodiments of a method of treating a cancer that expresses a mutant form of a RAS protein, the selective CDK9 inhibitor and the additional therapeutic agent are administered simultaneously. In some embodiments of a method of treating a cancer that expresses a mutant form of a RAS protein, the selective CDK9 inhibitor is administered before the additional therapeutic agent. In some embodiments of a method of treating a cancer that expresses a mutant form of a RAS protein, the selective CDK9 inhibitor is administered after the additional therapeutic agent. In some embodiments of a method of treating a cancer that expresses a mutant form of a RAS protein, the selective CDK9 inhibitor is a compound that is 5-fluoro-4-(4-fluoro-2-methoxyphenyl)-N-{4-[(S-methylsulfonimidoyl)methyl]pyridin-2-yl}pyridin-2-amine:

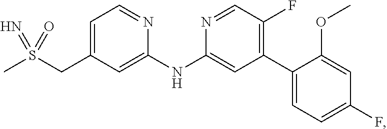 [SMILES: COc1cc(F)ccc1-c1cc(Nc2cc(CS(C)(=N)=O)ccn2)ncc1F]

or an enantiomer thereof, or a pharmaceutically acceptable salt or solvate thereof. In some embodiments of a method of treating a cancer that expresses a mutant form of a RAS protein, the enantiomer of the compound is the (+) 5-fluoro-4-(4-fluoro-2-methoxyphenyl)-N-{4-[(S-methylsulfonimidoyl)methyl]pyridin-2-yl}pyridin-2-amine. In some embodiments of a method of treating a cancer that expresses a mutant form of a RAS protein, the compound is administered at a dose of between about 1 mg and about 50 mg. In some embodiments of a method of treating a cancer that expresses a mutant form of a RAS protein, the compound is administered at a dose of between about 1 mg and about 30 mg. In some embodiments of a method of treating a cancer that expresses a mutant form of a RAS protein, the compound is administered at a dose of between about 10 mg and about 30 mg. In some embodiments of a method of treating a cancer that expresses a mutant form of a RAS protein, the compound is administered at a dose of between about 20 mg and about 30 mg. In some embodiments of a method of treating a cancer that expresses a mutant form of a RAS protein, the compound is administered at a dose of about 5 mg. In some embodiments of a method of treating a cancer that expresses a mutant form of a RAS protein, the compound is administered at a dose of about 10 mg. In some embodiments of a method of treating a cancer that expresses a mutant form of a RAS protein, the compound is administered at a dose of about 15 mg. In some embodiments of a method of treating a cancer that expresses a mutant form of a RAS protein, the compound is administered at a dose of about 20 mg. In some embodiments of a method of treating a cancer that expresses a mutant form of a RAS protein, the compound is administered at a dose of about 25 mg. In some embodiments of a method of treating a cancer that expresses a mutant form of a RAS protein, the compound is administered at a dose of about 30 mg. In some embodiments of a method of treating a cancer that expresses a mutant form of a RAS protein, the selective CDK9 inhibitor is KB-0742, PRT2527, or A-1592668. In some embodiments of a method of treating a cancer that expresses a mutant form of a RAS protein, the selective CDK9 inhibitor is KB-0742, PRT2527, A-1592668, AZD4573, or fadraciclib. In some embodiments of a method of treating a cancer that expresses a mutant form of a RAS protein, the selective CDK9 inhibitor is KB-0742. In some embodiments of a method of treating a cancer that expresses a mutant form of a RAS protein, the selective CDK9 inhibitor is PRT2527. In some embodiments of a method of treating a cancer that expresses a mutant form of a RAS protein, the selective CDK9 inhibitor is A-1592668. In some embodiments of a method of treating a cancer that expresses a mutant form of a RAS protein, the selective CDK9 inhibitor is AZD4573. In some embodiments of a method of treating a cancer that expresses a mutant form of a RAS protein, the selective CDK9 inhibitor is fadraciclib.

Also disclosed herein is a method of treating a cancer associated with a RAS mutation in a subject in need thereof, the method comprising administering to the subject in need a therapeutically-effective amount of a selective CDK9 inhibitor. In some embodiments of a method of treating a cancer associated with a RAS mutation, the RAS mutation is a NRAS mutation. In some embodiments of a method of treating a cancer associated with a RAS mutation, the RAS mutation is a HRAS mutation. In some embodiments of a method of treating a cancer associated with a RAS mutation, the RAS mutation is a KRAS mutation. In some embodiments of a method of treating a cancer associated with a RAS mutation, the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V. In some embodiments of a method of treating a cancer associated with a RAS mutation, the mutation comprises one or more amino acid substitutions selected from the group consisting of G12C, G12D, G12V, G13S, and Q61R. In some embodiments of a method of treating a cancer associated with a RAS mutation, the mutation comprises the amino acid substitution G12S. In some embodiments of a method of treating a cancer associated with a RAS mutation, the mutation comprises the amino acid substitution G12C. In some embodiments of a method of treating a cancer associated with a RAS mutation, the mutation comprises the amino acid substitution G12D. In some embodiments of a method of treating a cancer associated with a RAS mutation, the mutation comprises the amino acid substitution G12V. In some embodiments of a method of treating a cancer associated with a RAS mutation, the mutation comprises the amino acid substitution G13S. In some embodiments of a method of treating a cancer associated with a RAS mutation, the mutation comprises the amino acid substitution G13D. In some embodiments of a method of treating a cancer associated with a RAS mutation, the mutation comprises the amino acid substitution Q61R. In some embodiments of a method of treating a cancer associated with a RAS mutation, the mutation comprises the amino acid substitution Q61H. In some embodiments of a method of treating a cancer associated with a RAS mutation, the mutation comprises secondary adaptive RAS mutation. In some embodiments of a method of treating a cancer associated with a RAS mutation, the cancer is bladder cancer, brain cancer, brain metastases, breast cancer, cervical cancer, colorectal cancer, endometrial cancer, gastrointestinal cancer, glioblastoma, head and neck cancer, leukemias, liver cancer, lung cancer, lymphomas, melanoma, multiple myeloma, ovarian cancer, pancreatic cancer, peritoneal cancer, prostate cancer, renal cancer, salivary gland carcinoma, thyroid cancer, or uterine carcinoma. In some embodiments of a method of treating a cancer associated with a RAS mutation, the cancer is non-small cell lung cancer, pancreatic cancer, or glioblastoma. In some embodiments of a method of treating a cancer associated with a RAS mutation, the cancer is colorectal cancer, non-small cell lung cancer, or pancreatic cancer. In some embodiments of a method of treating a cancer associated with a RAS mutation, the cancer is glioblastoma. In some embodiments of a method of treating a cancer associated with a RAS mutation, the cancer is colorectal cancer. In some embodiments of a method of treating a cancer associated with a RAS mutation, the cancer is non-small cell lung cancer. In some embodiments of a method of treating a cancer associated with a RAS mutation, the cancer is pancreatic cancer. In some embodiments of a method of treating a cancer associated with a RAS mutation, the selective CDK9 inhibitor is administered in multiple 21-day cycles. In some embodiments of a method of treating a cancer associated with a RAS mutation, the selective CDK9 inhibitor is administered in at least one 21-day cycle. In some embodiments of a method of treating a cancer associated with a RAS mutation, the selective CDK9 inhibitor is administered in at least two 21-day cycles. In some embodiments of a method of treating a cancer associated with a RAS mutation, the selective CDK9 inhibitor is administered in at least three 21-day cycles. In some embodiments of a method of treating a cancer associated with a RAS mutation, the selective CDK9 inhibitor is administered in at least four 21-day cycles. In some embodiments of a method of treating a cancer associated with a RAS mutation, the selective CDK9 inhibitor is administered in at least five 21-day cycles. In some embodiments of a method of treating a cancer associated with a RAS mutation, the compound is administered in at least six 21-day cycles. In some embodiments of a method of treating a cancer associated with a RAS mutation, the selective CDK9 inhibitor is administered in at least 12 21-day cycles. In some embodiments of a method of treating a cancer associated with a RAS mutation, the selective CDK9 inhibitor is administered once weekly. In some embodiments of a method of treating a cancer associated with a RAS mutation, the selective CDK9 inhibitor is administered twice weekly. In some embodiments of a method of treating a cancer associated with a RAS mutation, the selective CDK9 inhibitor is administered weekly three days on and four days off. In some embodiments of a method of treating a cancer associated with a RAS mutation, the method further comprises administering an additional therapeutic agent. In some embodiments of a method of treating a cancer associated with a RAS mutation, the additional therapeutic agent is an AKT inhibitor, BCL2 inhibitor, BTK inhibitor, EGFR inhibitor, farnesyl transferase inhibitor, HER2 inhibitor, MEK inhibitor, mTOR inhibitor, PI3K inhibitor, RAF inhibitor, RAS inhibitor, or any combinations thereof. In some embodiments of a method of treating a cancer associated with a RAS mutation, the selective CDK9 inhibitor and the additional therapeutic agent are administered sequentially. In some embodiments of a method of treating a cancer associated with a RAS mutation, the selective CDK9 inhibitor and the additional therapeutic agent are administered simultaneously. In some embodiments of a method of treating a cancer associated with a RAS mutation, the selective CDK9 inhibitor is administered before the additional therapeutic agent. In some embodiments of a method of treating a cancer associated with a RAS mutation, the selective CDK9 inhibitor is administered after the additional therapeutic agent. In some embodiments of a method of treating a cancer associated with a RAS mutation, the selective CDK9 inhibitor is a compound that is 5-fluoro-4-(4-fluoro-2-methoxyphenyl)-N-{4-[(S-methylsulfonimidoyl)methyl]pyridin-2-yl}pyridin-2-amine:

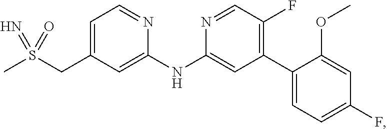 [SMILES: COc1cc(F)ccc1-c1cc(Nc2cc(CS(C)(=N)=O)ccn2)ncc1F]

or an enantiomer thereof, or a pharmaceutically acceptable salt or solvate thereof. In some embodiments of a method of treating a cancer associated with a RAS mutation, the enantiomer of the compound is the (+) 5-fluoro-4-(4-fluoro-2-methoxyphenyl)-N-{4-[(S-methylsulfonimidoyl)methyl]pyridin-2-yl}pyridin-2-amine.

In some embodiments of a method of treating a cancer associated with a RAS mutation, the compound is administered at a dose of between about 1 mg and about 50 mg. In some embodiments of a method of treating a cancer associated with a RAS mutation, the compound is administered at a dose of between about 1 mg and about 30 mg. In some embodiments of a method of treating a cancer associated with a RAS mutation, the compound is administered at a dose of between about 10 mg and about 30 mg. In some embodiments of a method of treating a cancer associated with a RAS mutation, the compound is administered at a dose of between about 20 mg and about 30 mg. In some embodiments of a method of treating a cancer associated with a RAS mutation, the compound is administered at a dose of about 5 mg. In some embodiments of a method of treating a cancer associated with a RAS mutation, the compound is administered at a dose of about 10 mg. In some embodiments of a method of treating a cancer associated with a RAS mutation, the compound is administered at a dose of about 15 mg. In some embodiments of a method of treating a cancer associated with a RAS mutation, the compound is administered at a dose of about 20 mg. In some embodiments of a method of treating a cancer associated with a RAS mutation, the compound is administered at a dose of about 25 mg. In some embodiments of a method of treating a cancer associated with a RAS mutation, the compound is administered at a dose of about 30 mg. In some embodiments of a method of treating a cancer associated with a RAS mutation, the selective CDK9 inhibitor is KB-0742, PRT2527, or A-1592668. In some embodiments of a method of treating a cancer associated with a RAS mutation, the selective CDK9 inhibitor is KB-0742, PRT2527, A-1592668, AZD4573, or fadraciclib. In some embodiments of a method of treating a cancer associated with a RAS mutation, the selective CDK9 inhibitor is KB-0742. In some embodiments of a method of treating a cancer associated with a RAS mutation, the selective CDK9 inhibitor is PRT2527. In some embodiments of a method of treating a cancer associated with a RAS mutation, the selective CDK9 inhibitor is A-1592668.

Also disclosed herein is a method for treating cancer that expresses a mutant form of a RAS protein in a subject in need thereof with a selective CDK9 inhibitor, the method comprising:
(a) receiving a biological sample from the subject;
(b) assaying the biological sample for one or more RAS mutations;
(c) determining if one or more RAS mutations are present in the biological sample;
(d) employing the determination of one or more RAS mutations in the biological sample to predict responsiveness of the subject in need thereof to the compound, wherein the presence of one or more RAS mutations in the biological sample predicts that the subject will be responsive to the selective CDK9 inhibitor; and
(e) administering a therapeutically-effective amount of the selective CDK9 inhibitor to the subject.

Also disclosed herein is a method of selecting a subject with cancer for treatment with a selective CDK9 inhibitor, the method comprising:
(a) receiving a biological sample from the subject with cancer; and
(b) determining if one or more RAS mutations are present in the biological sample.

Also disclosed herein is a method for selecting a subject with cancer for treatment with a selective CDK9 inhibitor, the method comprising:
(a) receiving a biological sample from the subject;
(b) assaying the biological sample for one or more RAS mutations;
(c) determining if one or more RAS mutations are present in the biological sample;
(d) employing the determination of one or more RAS mutations in the biological sample to predict responsiveness of the subject in need thereof to the compound, wherein the presence of one or more RAS mutations in the biological sample predicts that the subject will be responsive to the selective CDK9 inhibitor; and
(e) administering a therapeutically-effective amount of the selective CDK9 inhibitor to the subject

Also disclosed herein is a method of predicting a therapeutic response for the treatment of cancer to a selective CDK9 inhibitor in a subject with cancer, the method comprising:
(a) determining if one or more RAS mutations are present in a biological sample obtained from the subject with cancer; and
(b) determining whether the subject is likely to respond administration of the selective CDK9 inhibitor.

Also disclosed herein is a method of contacting a cell with a selective CDK9 inhibitor, the method comprising:
(a) receiving a cell;
(b) assaying the cell for one or more RAS mutations;
(c) determining if one or more RAS mutations are present in the cell;
(d) employing the determination of one or more RAS mutations in the cell to predict responsiveness of the cell to the selective CDK9 inhibitor, wherein the presence of one or more RAS mutations in the cell predicts that the cell will be responsive to the selective CDK9 inhibitor; and
(e) selectively contacting a cell predicted to be responsive to the selective CDK9 inhibitor with the selective CDK9 inhibitor.

In some embodiments, the biological sample is a tumor biopsy. In some embodiments, the biological sample is an aspirate. In some embodiments, the RAS mutation is a NRAS mutation. In some embodiments, the RAS mutation is a HRAS mutation. In some embodiments, the RAS mutation is a KRAS mutation. In some embodiments, the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V. In some embodiments, the mutation comprises one or more amino acid substitutions selected from the group consisting of G12C, G12D, G12V, G13S, and Q61R. In some embodiments, the mutation comprises the amino acid substitution G12S. In some embodiments, the mutation comprises the amino acid substitution G12C. In some embodiments, the mutation comprises the amino acid substitution G12D. In some embodiments, the mutation comprises the amino acid substitution G12V. In some embodiments, the mutation comprises the amino acid substitution G13S. In some embodiments, the mutation comprises the amino acid substitution G13D. In some embodiments, the mutation comprises the amino acid substitution Q61R. In some embodiments, the mutation comprises the amino acid substitution Q61H. In some embodiments, the mutation comprises a secondary adaptive RAS mutation. In some embodiments, the cancer is bladder cancer, brain cancer, brain metastases, breast cancer, cervical cancer, colorectal cancer, endometrial cancer, gastrointestinal cancer, glioblastoma, head and neck cancer, leukemias, liver cancer, lung cancer, lymphomas, melanoma, multiple myeloma, ovarian cancer, pancreatic cancer, peritoneal cancer, prostate cancer, renal cancer, salivary gland carcinoma, thyroid cancer, or uterine carcinoma. In some embodiments, the cancer is non-small cell lung cancer, pancreatic cancer, or glioblastoma. In some embodiments, the cancer is colorectal cancer, non-small cell lung cancer, or pancreatic cancer. In some embodiments, the cancer is glioblastoma. In some embodiments, the cancer is colorectal cancer. In some embodiments, the cancer is non-small cell lung cancer. In some embodiments, the cancer is pancreatic cancer. In some embodiments, the selective CDK9 inhibitor is administered in multiple 21-day cycles. In some embodiments, the selective CDK9 inhibitor is administered in at least one 21-day cycle. In some embodiments, the selective CDK9 inhibitor is administered in at least two 21-day cycles. In some embodiments, the selective CDK9 inhibitor is administered in at least three 21-day cycles. In some embodiments, the selective CDK9 inhibitor is administered in at least four 21-day cycles. In some embodiments, the selective CDK9 inhibitor is administered in at least five 21-day cycles. In some embodiments, the compound is administered in at least six 21-day cycles. In some embodiments, the selective CDK9 inhibitor is administered in at least 12 21-day cycles. In some embodiments, the selective CDK9 inhibitor is administered once weekly. In some embodiments, the selective CDK9 inhibitor is administered twice weekly. In some embodiments, the selective CDK9 inhibitor is administered weekly three days on and four days off. In some embodiments, the method further comprises administering an additional therapeutic agent. In some embodiments, the additional therapeutic agent is an AKT inhibitor, BCL2 inhibitor, BTK inhibitor, EGFR inhibitor, farnesyl transferase inhibitor, HER2 inhibitor, MEK inhibitor, mTOR inhibitor, PI3K inhibitor, RAF inhibitor, RAS inhibitor, or any combinations thereof. In some embodiments, the selective CDK9 inhibitor and the additional therapeutic agent are administered sequentially. In some embodiments, the selective CDK9 inhibitor and the additional therapeutic agent are administered simultaneously. In some embodiments, the selective CDK9 inhibitor is administered before the additional therapeutic agent. In some embodiments, the selective CDK9 inhibitor is administered after the additional therapeutic agent. In some embodiments, the selective CDK9 inhibitor is a compound that is 5-fluoro-4-(4-fluoro-2-methoxyphenyl)-N-{4-[(S-methylsulfonimidoyl)methyl]pyridin-2-yl}pyridin-2-amine:

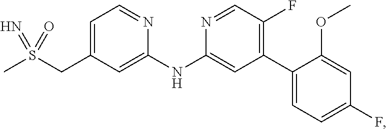 [SMILES: COc1cc(F)ccc1-c1cc(Nc2cc(CS(C)(=N)=O)ccn2)ncc1F]

or an enantiomer thereof, or a pharmaceutically acceptable salt or solvate thereof. In some embodiments, the enantiomer of the compound is the (+) 5-fluoro-4-(4-fluoro-2-methoxyphenyl)-N-{4-[(S-methylsulfonimidoyl)methyl]pyridin-2-yl}pyridin-2-amine. In some embodiments, the compound is administered at a dose of between about 1 mg and about 50 mg. In some embodiments, the compound is administered at a dose of between about 1 mg and about 30 mg. In some embodiments, the compound is administered at a dose of between about 10 mg and about 30 mg. In some embodiments, the compound is administered at a dose of between about 20 mg and about 30 mg. In some embodiments, the compound is administered at a dose of about 5 mg. In some embodiments, the compound is administered at a dose of about 10 mg. In some embodiments, the compound is administered at a dose of about 15 mg. In some embodiments, the compound is administered at a dose of about 20 mg. In some embodiments, the compound is administered at a dose of about 25 mg. In some embodiments, the compound is administered at a dose of about 30 mg. In some embodiments, the selective CDK9 inhibitor is KB-0742, PRT2527, or A-1592668. In some embodiments, the selective CDK9 inhibitor is KB-0742, PRT2527, A-1592668, AZD4573, or fadraciclib. In some embodiments, the selective CDK9 inhibitor is KB-0742. In some embodiments, the selective CDK9 inhibitor is PRT2527. In some embodiments, the selective CDK9 inhibitor is A-1592668.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows the KRAS cancer cell lines with compound 1′ IC50 values in the bottom quartile of the IC50 range which are the most sensitive to CDK9 inhibition (≤0.0905 μM; N=110) compared to the KRAS cancer cell lines in the top quartile (≥0.1520 μM; N=130) which are the least sensitive to compound 1′. The y axis denotes the count of the number of cell lines in each the bottom and top quartile.

FIG. 2 shows the NRAS cancer cell lines with compound 1′ IC50 values in the bottom quartile of the IC50 range which are the most sensitive to CDK9 inhibition (≤0.0905 μM; N=110) compared to the NRAS cancer cell lines in the top quartile (≥0.1520 μM; N=130) which are the least sensitive to compound 1′. The y axis denotes the count of the number of cell lines in each the bottom and top quartile.

FIG. 3 shows the HRAS cancer cell lines with compound 1′ IC50 values in the bottom quartile of the IC50 range which are the most sensitive to CDK9 inhibition (≤0.0905 μM; N=110) compared to the HRAS cancer cell lines in the top quartile (≥0.1520 μM; N=130) which are the least sensitive to compound 1′. The y axis denotes the count of the number of cell lines in each the bottom and top quartile.

### DETAILED DESCRIPTION OF THE INVENTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent or improve an unwanted condition or disease of a subject. In some embodiments, a therapeutic agent such as a compound 1 is directed to the treatment and/or the amelioration of cancers.

“Administering” when used in conjunction with a therapeutic means to administer a therapeutic systemically or locally, as directly into or onto a target tissue, or to administer a therapeutic to a subject whereby the therapeutic positively impacts the tissue to which it is targeted. Thus, as used herein, the term “administering”, when used in conjunction with a composition described herein, can include, but is not limited to, providing a composition into or onto the target tissue; providing a composition systemically to a subject by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the subject is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable”, it is meant the carrier, diluent or excipient must be compatible with the other ingredients of the formulation and not deleterious to the recipient subject thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to therapeutic treatment, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition.

For the purposes of the present invention, the substituents have the following meaning, unless otherwise specified:

The term “halogen”, “halogen atom” or “halo” represents fluorine, chlorine, bromine and iodine, particularly bromine, chlorine or fluorine, preferably chlorine or fluorine, more preferably fluorine.

The term “alkyl-” represents a linear or branched alkyl- group having the number of carbon atoms specifically indicated, e.g. C₁-C₁₀ one, two, three, four, five, six, seven, eight, nine or ten carbon atoms, e.g. methyl-, ethyl-, n-propyl-, isopropyl-, n-butyl-, isobutyl-, sec-butyl-, tert-butyl-, pentyl-, isopentyl-, hexyl-, heptyl-, octyl-, nonyl-, decyl-, 2-methylbutyl-, 1-methylbutyl-, 1-ethylpropyl-, 1,2-dimethylpropyl-, neo-pentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, or 1,2-dimethylbutyl-. If the number of carbon atoms is not specifically indicated the term “alkyl-” represents a linear or branched alkyl- group having, as a rule, 1 to 9, particularly 1 to 6, preferably 1 to 4 carbon atoms. Particularly, the alkyl- group has 1, 2, 3, 4, 5 or 6 carbon atoms (“C₁-C₆-alkyl-”), e.g. methyl-, ethyl-, n-propyl-, isopropyl-, n-butyl-, tert-butyl-, pentyl-, isopentyl-, hexyl-, 2-methylbutyl-, 1-methylbutyl-, 1-ethylpropyl-, 1,2-dimethylpropyl-, neo-pentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, or 1,2-dimethylbutyl-. Preferably, the alkyl- group has 1, 2 or 3 carbon atoms (“C₁-C₃-alkyl-”), methyl-, ethyl-, n-propyl- or isopropyl-.

The term “C₂-C₈-alkylene” is to be understood as preferably meaning a linear, bivalent and saturated hydrocarbon group having 2 to 6, particularly 2, 3, 4 or 5 carbon atoms, as in “C₂-C₅-alkylene”, more particularly 2, 3 or 4 carbon atoms, as in “C₂-C₄-alkylene” e.g. ethylene, n-propylene, n-butylene, n-pentylene, or n-hexylene, preferably n-propylene or n-butylene.

The term “C₂-C₆-alkenyl-” is to be understood as preferably meaning a linear or branched, monovalent hydrocarbon group, which contains one double bond, and which has 2, 3, 4, 5 or 6 carbon atoms (“C₂-C₆-alkenyl-”). Particularly, said alkenyl- group is a C₂-C₃-alkenyl-, C₃-C₆-alkenyl- or C₃-C₄-alkenyl-group. Said alkenyl- group is, for example, a vinyl-, allyl-, (E)-2-methylvinyl-, (2)-2-methylvinyl- or isopropenyl- group.

The term “C₂-C₆-alkynyl-” is to be understood as preferably meaning a linear or branched, monovalent hydrocarbon group which contains one triple bond, and which contains 2, 3, 4, 5 or 6 carbon atoms. Particularly, said alkynyl- group is a C₂-C₃-alkynyl-, C₃-C₆-alkynyl- or C₃-C₄-alkynyl- group. Said C₂-C₃-alkynyl- group is, for example, an ethynyl-, prop-1-ynyl- or prop-2-ynyl- group.

The term “C₃-C₇-cycloalkyl-” is to be understood as preferably meaning a saturated or partially unsaturated, monovalent, monocyclic hydrocarbon ring which contains 3, 4, 5, 6 or 7 carbon atoms. Said C₃-C₇-cycloalkyl- group is for example, a monocyclic hydrocarbon ring, e.g. a cyclopropyl-, cyclobutyl-, cyclopentyl-, cyclohexyl- or cycloheptyl- group. Said cycloalkyl- ring is non-aromatic but can optionally contain one or more double bonds e.g. cycloalkenyl-, such as a cyclopropenyl-, cyclobutenyl-, cyclopentenyl-, cyclohexenyl- or cycloheptenyl- group, wherein the bond between said ring with the rest of the molecule may be to any carbon atom of said ring, be it saturated or unsaturated. Particularly, said cycloalkyl- group is a C₄-C₆-cycloalkyl-, a C₅-C₆-cycloalkyl- or a cyclohexyl- group.

The term “C₃-C₅-cycloalkyl-” is to be understood as preferably meaning a saturated, monovalent, monocyclic hydrocarbon ring which contains 3, 4 or 5 carbon atoms. In particular said C₃-C₅-cycloalkyl-group is a monocyclic hydrocarbon ring such as a cyclopropyl-, cyclobutyl- or cyclopentyl- group. Preferably said “C₃-C₅-cycloalkyl-” group is a cyclopropyl- group.

The term “C₃-C₄-cycloalkyl-” is to be understood as preferably meaning a saturated, monovalent, monocyclic hydrocarbon ring which contains 3 or 4 carbon atoms. In particular, said C₃-C₄-cycloalkyl- group is a monocyclic hydrocarbon ring such as a cyclopropyl- or cyclobutyl- group.

The term “heterocyclyl-” is to be understood as meaning a saturated or partially unsaturated, monovalent, mono- or bicyclic hydrocarbon ring which contains 3, 4, 5, 6, 7, 8 or 9 carbon atoms and further containing 1, 2 or 3 heteroatom-containing groups selected from oxygen, sulfur, nitrogen. Particularly, the term “heterocyclyl-” is to be understood as meaning a “4- to 10-membered heterocyclic ring”.

The term “a 4- to 10-membered heterocyclic ring” is to be understood as meaning a saturated or partially unsaturated, monovalent, mono- or bicyclic hydrocarbon ring which contains 3, 4, 5, 6, 7, 8 or 9 carbon atoms, and further containing 1, 2 or 3 heteroatom-containing groups selected from oxygen, sulfur, nitrogen.

A C₃-C₉-heterocyclyl- is to be understood as meaning a heterocyclyl- which contains at least 3, 4, 5, 6, 7, 8 or 9 carbon atoms and additionally at least one heteroatom as ring atoms. Accordingly in case of one heteroatom the ring is 4- to 10-membered, in case of two heteroatoms the ring is 5- to 11-membered and in case of three heteroatoms the ring is 6- to 12-membered.

Said heterocyclic ring is for example, a monocyclic heterocyclic ring such as an oxetanyl-, azetidinyl-, tetrahydrofuranyl-, pyrrolidinyl-, 1,3-dioxolanyl-, imidazolidinyl-, pyrazolidinyl-, oxazolidinyl-, isoxazolidinyl-, 1,4-dioxanyl-, pyrrolinyl-, tetrahydropyranyl-, piperidinyl-, morpholinyl-, 1,3-dithianyl-, thiomorpholinyl-, piperazinyl-, or chinuclidinyl- group. Optionally, said heterocyclic ring can contain one or more double bonds, e.g. 4H-pyranyl-, 2H-pyranyl-, 2,5-dihydro-1H-pyrrolyl-, 1,3-dioxolyl-, 4H-1,3,4-thiadiazinyl-, 2,5-dihydrofuranyl-, 2,3-dihydrofuranyl-, 2,5-dihydrothienyl-, 2,3-dihydrothienyl-, 4,5-dihydrooxazolyl-, 4,5-dihydroisoxazolyl-, or 4H-1,4-thiazinyl- group, or, it may be benzo fused.

Particularly, a C₃-C₇-heterocyclyl- is to be understood as meaning a heterocyclyl- which contains at least 3, 4, 5, 6, or 7 carbon atoms and additionally at least one heteroatom as ring atoms. Accordingly in case of one heteroatom the ring is 4- to 8-membered, in case of two heteroatoms the ring is 5- to 9-membered and in case of three heteroatoms the ring is 6- to 10-membered.

Particularly, a C₃-C₆-heterocyclyl- is to be understood as meaning a heterocyclyl- which contains at least 3, 4, 5 or 6 carbon atoms and additionally at least one heteroatom as ring atoms. Accordingly in case of one heteroatom the ring is 4- to 7-membered, in case of two heteroatoms the ring is 5- to 8-membered and in case of three heteroatoms the ring is 6- to 9-membered.

Particularly, the term “heterocyclyl-” is to be understood as being a heterocyclic ring which contains 3, 4 or 5 carbon atoms, and 1, 2 or 3 of the above-mentioned heteroatom-containing groups (a “4- to 8-membered heterocyclic ring”), more particularly said ring can contain 4 or 5 carbon atoms, and 1, 2 or 3 of the above-mentioned heteroatom-containing groups (a “5- to 8-membered heterocyclic ring”), more particularly said heterocyclic ring is a “6-membered heterocyclic ring”, which is to be understood as containing 4 carbon atoms and 2 of the above-mentioned heteroatom-containing groups or 5 carbon atoms and one of the above-mentioned heteroatom-containing groups, preferably 4 carbon atoms and 2 of the above-mentioned heteroatom-containing groups.

The term “C₁-C₆-alkoxy-” is to be understood as preferably meaning a linear or branched, saturated, monovalent, hydrocarbon group of formula —O-alkyl-, in which the term “alkyl-” is defined supra, e.g. a methoxy, ethoxy, n-propoxy, iso-propoxy, n-butoxy, iso-butoxy, tert-butoxy, sec-butoxy, pentyloxy, iso-pentyloxy, n-hexyloxy group, or an isomer thereof. Particularly, the “C₁-C₆-alkoxy-” group is a “C₁-C₄-alkoxy-”, a “C₁-C₃-alkoxy-”, a methoxy, ethoxy, or propoxy group, preferably a methoxy, ethoxy or propoxy group. Further preferred is a “C₁-C₂-alkoxy-” group, particularly a methoxy or ethoxy group.

The term “C₁-C₃-fluoroalkoxy-” is to be understood as preferably meaning a linear or branched, saturated, monovalent, C₁-C₃-alkoxy- group, as defined supra, in which one or more of the hydrogen atoms is replaced, identically or differently, by one or more fluorine atoms. Said C₁-C₃-fluoroalkoxy- group is, for example a 1,1-difluoromethoxy-, a 1,1,1-trifluoromethoxy-, a 2-fluoroethoxy-, a 3-fluoropropoxy-, a 2,2,2-trifluoroethoxy-, a 3,3,3-trifluoropropoxy-, particularly a “C₁-C₂-fluoroalkoxy-” group.

The term “alkylamino-” is to be understood as preferably meaning an alkylamino group with one linear or branched alkyl- group as defined supra. (C₁-C₃)-alkylamino- for example means a monoalkylamino group with 1, 2 oder 3 carbon atoms, (C₁-C₆)-alkylamino- with 1, 2, 3, 4, 5 or 6 carbon atoms. The term “alkylamino-” comprises for example methylamino-, ethylamino-, n-propylamino-, iso-propylamino-, tert.-butylamino-, n-pentylamino- or n-hexylamino-.

The term “dialkylamino-” is to be understood as preferably meaning an alkylamino group having two linear or branched alkyl- groups as defined supra, which are independent from each other. (C₁-C₃)-dialkylamino- for example represents a dialkylamino group with two alkyl groups each of them having 1 to 3 carbon atoms per alkyl group. The term “dialkylamino-” comprises for example: N,N-dimethylamino-, N,N-diethylamino-, N-ethyl-N-methylamino-, N-methyl-N-n-propylamino-, N-iso-propyl-N-n-propylamino-, N-tert-butyl-N-methylamino-, N-ethyl-N-n-pentylamino- and N-n-hexyl-N-methylamino-.

The term “cyclic amine” is to be understood as preferably meaning a cyclic amine group. Preferably, a cyclic amine means a saturated, monocyclic group with 4 to 10, preferably 4 to 7 ring atoms of which at least one ring atom is a nitrogen atom. Suitable cyclic amines are especially azetidine, pyrrolidine, piperidine, piperazine, 1-methylpiperazine, morpholine, thiomorpholine, which could be optionally substituted by one or two methyl- groups.

The term “halo-C₁-C₃-alkyl-”, or, used synonymously, “C₁-C₃-haloalkyl-”, is to be understood as preferably meaning a linear or branched, saturated, monovalent hydrocarbon group in which the term “C₁-C₃-alkyl-” is defined supra, and in which one or more hydrogen atoms is replaced by a halogen atom, identically or differently, i.e. one halogen atom being independent from another. Preferably, a halo-C₁-C₃-alkyl- group is a fluoro-C₁-C₃-alkyl- or a fluoro-C₁-C₂-alkyl- group, such as for example —CF₃, —CHF₂, —CH₂F, —CF₂CF₃, or —CH₂CF₃, more preferably it is —CF₃.

The term “hydroxy-C₁-C₃-alkyl-”, is to be understood as preferably meaning a linear or branched, saturated, monovalent hydrocarbon group in which the term “C₁-C₃-alkyl-” is defined supra, and in which one or more hydrogen atoms is replaced by hydroxy group, preferably not more than one hydrogen atom per carbon atom being replaced by a hydroxy group. Particularly, a hydroxy-C₁-C₃-alkyl- group is, for example, —CH₂OH, —CH₂—CH₂OH, —C(H)OH—CH₂OH, —CH₂—CH₂—CH₂OH.

The term “phenyl-C₁-C₃-alkyl-” is to be understood as preferably meaning a phenyl- group, in which one of the hydrogen atoms is replaced by a C₁-C₃-alkyl- group, as defined supra, which links the phenyl-C₁-C₃-alkyl- group to the rest of the molecule. Particularly, the “phenyl-C₁-C₃-alkyl-” is a phenyl-C₁-C₂-alkyl-, preferably it is a benzyl- group.

The term “heteroaryl-” is to be understood as preferably meaning a monovalent, aromatic ring system having 5, 6, 7, 8, 9, 10, 11, 12, 13 or 14 ring atoms (a “5- to 14-membered heteroaryl-” group), particularly 5 (a “5-membered heteroaryl-”) or 6 (a “6-membered heteroaryl-”) or 9 (a “9-membered heteroaryl-”) or 10 ring atoms (a “10-membered heteroaryl-”), and which contains at least one heteroatom which may be identical or different, said heteroatom being such as oxygen, nitrogen or sulfur, and can be monocyclic, bicyclic, or tricyclic, and in addition in each case can be benzo-condensed. Particularly, heteroaryl- is selected from thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, tetrazolyl- etc., and benzo derivatives thereof, such as, for example, benzofuranyl-, benzothienyl-, benzoxazolyl-, benzisoxazolyl-, benzimidazolyl-, benzotriazolyl-, indazolyl-, indolyl-, isoindolyl-, etc.; or pyridyl-, pyridazinyl-, pyrimidinyl-, pyrazinyl-, triazinyl-, etc., and benzo derivatives thereof, such as, for example, quinolinyl-, quinazolinyl-, isoquinolinyl-, etc.; or azocinyl-, indolizinyl-, purinyl-, etc., and benzo derivatives thereof; or cinnolinyl-, phthalazinyl-, quinazolinyl-, quinoxalinyl-, naphthyridinyl-, pteridinyl-, carbazolyl-, acridinyl-, phenazinyl-, phenothiazinyl-, phenoxazinyl-, xanthenyl-, or oxepinyl-, etc. Preferably, heteroaryl- is selected from monocyclic heteroaryl-, 5-membered heteroaryl- or 6-membered heteroaryl-.

The term “5-membered heteroaryl-” is understood as preferably meaning a monovalent, aromatic ring system having 5 ring atoms and which contains at least one heteroatom which may be identical or different, said heteroatom being such as oxygen, nitrogen or sulfur. Particularly, “5-membered heteroaryl-” is selected from thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, tetrazolyl-.

The term “6-membered heteroaryl-” is understood as preferably meaning a monovalent, aromatic ring system having 6 ring atoms and which contains at least one heteroatom which may be identical or different, said heteroatom being such as oxygen, nitrogen or sulfur. Particularly, “6-membered heteroaryl-” is selected from pyridyl-, pyridazinyl-, pyrimidinyl-, pyrazinyl-, triazinyl-.

The term “heteroaryl-C₁-C₃-alkyl-” is to be understood as preferably meaning a heteroaryl-, a 5-membered heteroaryl- or a 6-membered heteroaryl- group, each as defined supra, in which one of the hydrogen atoms is replaced by a C₁-C₃-alkyl- group, as defined supra, which links the heteroaryl-C₁-C₃-alkyl- group to the rest of the molecule. Particularly, the “heteroaryl-C₁-C₃-alkyl-” is a heteroaryl-C₁-C₆-alkyl-, a pyridinyl-C₁-C₃-alkyl-, a pyridinylmethyl-, a pyridinylethyl-, a pyridinylpropyl-, a pyrimidinyl-C₁-C₃-alkyl-, a pyrimidinylmethyl-, a pyrimidinylethyl-, a pyrimidinylpropyl-, preferably a pyridinylmethyl- or a pyridinylethyl- or a pyrimidinylethyl- or a pyrimidinylpropyl- group.

As used herein, the term “C₁-C₃-alkylbenzene” refers to a partially aromatic hydrocarbon consisting of a benzene ring which is substituted by one or two C₁-C₃-alkyl groups, as defined supra. Particularly, “C₁-C₃-alkylbenzene” is toluene, ethylbenzene, cumene, n-propylbenzene, ortho-xylene, meta-xylene or para-xylene. Preferably, “C₁-C₃-alkylbenzene” is toluene.

### Selective CDK9 Inhibitors

Disclosed herein is a method of treating RAS mutant cancers in subjects in need thereof with a selective CDK9 inhibitor. In some embodiments the selective CDK9 inhibitor is compound 1, compound 1′, KB-0742, PRT2527, or A-1592668. In some embodiments the selective CDK9 inhibitor is KB-0742, PRT2527, A-1592668, AZD4573, or fadraciclib. In some embodiments the selective CDK9 inhibitor is a compound of Formula (I), Formula (II), Formula (III), Formula (IV), or Formula (V).

### Compound 1

Disclosed herein is a selective CDK9 inhibitor that is 5-fluoro-4-(4-fluoro-2-methoxyphenyl)-N-{4-[(S-methylsulfonimidoyl)methyl]pyridin-2-yl}pyridin-2-amine:

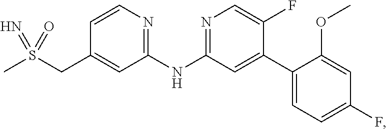 [SMILES: COc1cc(F)ccc1-c1cc(Nc2cc(CS(C)(=N)=O)ccn2)ncc1F]

or an enantiomer thereof, or a pharmaceutically acceptable salt or solvate thereof.

### Compound 1′

Disclosed herein is a selective CDK9 inhibitor that is (+)5-fluoro-4-(4-fluoro-2-methoxyphenyl)-N-{4-[(S-methylsulfonimidoyl)methyl]pyridin-2-yl}pyridin-2-amine:

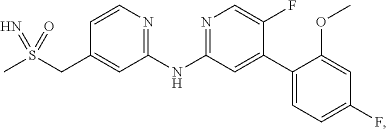 [SMILES: COc1cc(F)ccc1-c1cc(Nc2cc(CS(C)(=N)=O)ccn2)ncc1F]

or a pharmaceutically acceptable salt or solvate thereof.

Disclosed herein is a selective CDK9 inhibitor that is a compound of Formula (I):

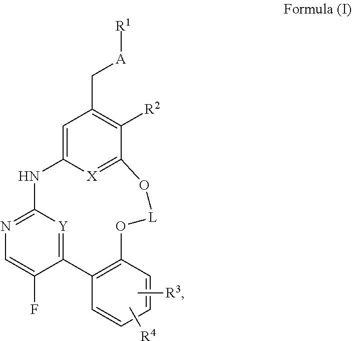

wherein:
A is a bivalent group selected from the group consisting of —S—, —S(═O)—, —S(═O)₂—, or —S(═O)(═NR⁵)—;
L is a C₂-C₆-alkylene group, wherein said group is optionally substituted with
   (i) one substituent selected from hydroxy, C₂-C₃-alkenyl, C₂-C₃-alkynyl, C₃-C₄-cycloalkyl, hydroxy-C₁-C₃-alkyl, and —(CH₂)NR⁶R⁷, and/or
   (ii) one or two or three substituents, identically or differently, selected from halogen and C₁-C₃-alkyl,
with the proviso that a C₂-alkylene group is not substituted with a hydroxy group,
or wherein one carbon atom of said C₂-C₆-alkylene group forms a three- or four-membered ring together with a bivalent group to which it is attached, wherein said bivalent group is selected from —CH₂CH₂—, —CH₂CH₂CH₂—, and —CH₂OCH₂—;
X, Y are CH or N with the proviso that one of X and Y represents CH and one of X and Y represents N;
R¹ is C₁-C₆-alkyl-, C₃-C₆-alkenyl, C₃-C₆-alkynyl, C₃-C₇-cycloalkyl-, heterocyclyl-, phenyl, heteroaryl, phenyl-C₁-C₃-alkyl-, or heteroaryl-C₁-C₃-alkyl-, wherein said group is optionally substituted with one or two or three substituents, identically or differently, selected from the group consisting of hydroxy, cyano, halogen, C₁-C₆-alkyl-, halo-C₁-C₃-alkyl-, C₁-C₆-alkoxy-, C₁-C₃-fluoroalkoxy-, —NH₂, alkylamino-, dialkylamino-, acetylamino-, N-methyl-N-acetylamino-, cyclic amines, —OP(═O)(OH)₂, —C(═O)OH, and —C(═O)NH₂;
R² is a hydrogen atom, a fluoro atom, a chloro atom, a bromo atom, cyano, C₁-C₃-alkyl-, C₁-C₃-alkoxy-, halo-C₁-C₃-alkyl-, or C₁-C₃-fluoroalkoxy-;
R³, R⁴ are, independently from each other, a hydrogen atom, a fluoro atom, a chloro atom, a bromo atom, cyano, C₁-C₃-alkyl-, C₁-C₃-alkoxy-, or halo-C₁-C₃-alkyl-, or C₁-C₃-fluoroalkoxy-;
R⁵ is a hydrogen atom, cyano, —C(═O)R⁸, —C(═O)OR⁸, —S(═O)₂R⁸, —C(═O)NR⁶R⁷, C₁-C₆-alkyl-, C₃-C₇ cycloalkyl-, heterocyclyl-, phenyl, heteroaryl, wherein said C₁-C₆-alkyl, C₃-C₇-cycloalkyl-, heterocyclyl-, phenyl or heteroaryl group is optionally substituted with one, two or three substituents, identically or differently, selected from the group consisting of halogen, hydroxy, cyano, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-;
R⁶, R⁷ is independently from each other, a group selected from a hydrogen atom, C₁-C₆-alkyl-, C₃-C₇-cycloalkyl-, heterocyclyl-, phenyl, benzyl, and heteroaryl, wherein said C₁-C₆-alkyl-, C₁-C₃-cycloalkyl-, heterocyclyl-, phenyl, benzyl or heteroaryl group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, a halo-C₁-C₃-alkyl-, and C₁-C₃-fluoroalkoxy-, or
R⁶ and R⁷, together with the nitrogen atom they are attached to, form a cyclic amine;
R⁸ is C₁-C₆-alkyl-, halo-C₁-C₃-alkyl-, C₃-C₇-cycloalkyl-, heterocyclyl-, phenyl, benzyl, or heteroaryl, wherein said group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-,
or an enantiomer, diastereomer, salt, solvate or salt of solvate thereof.

In some embodiments, the compound of Formula (I), or an enantiomer, diastereomer, salt, solvate or salt of solvate thereof is:

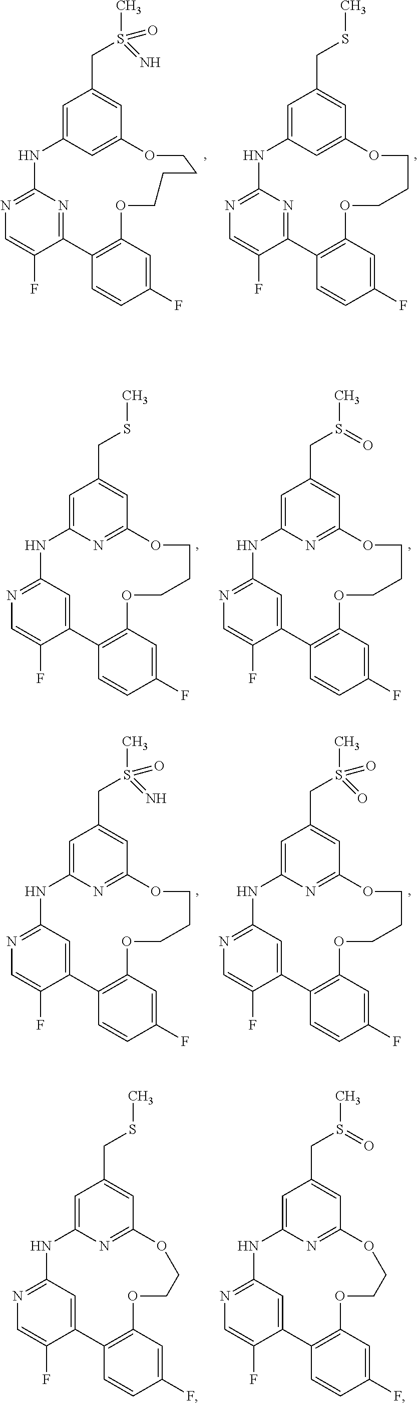

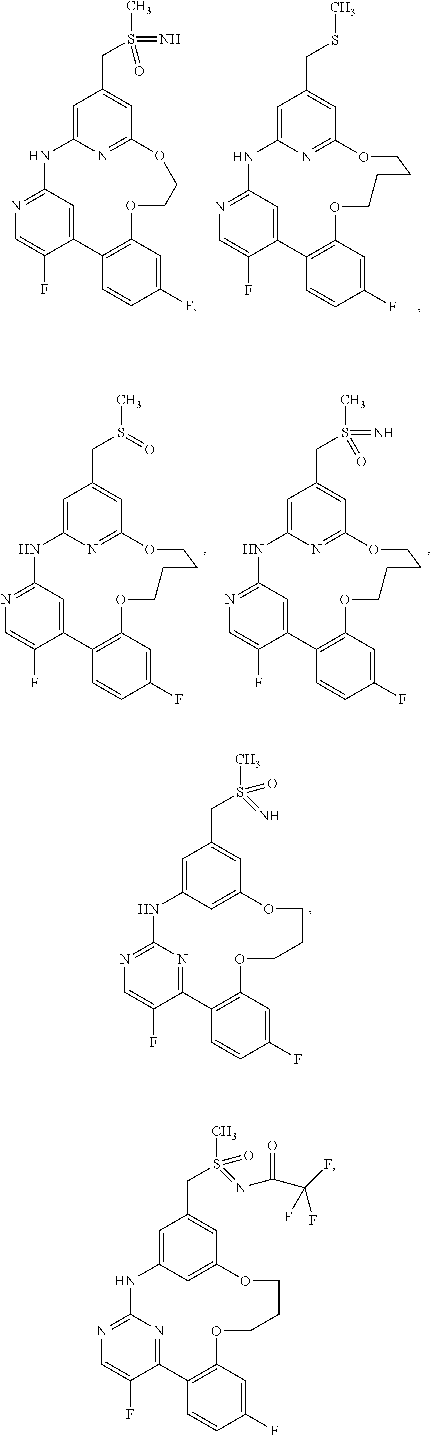

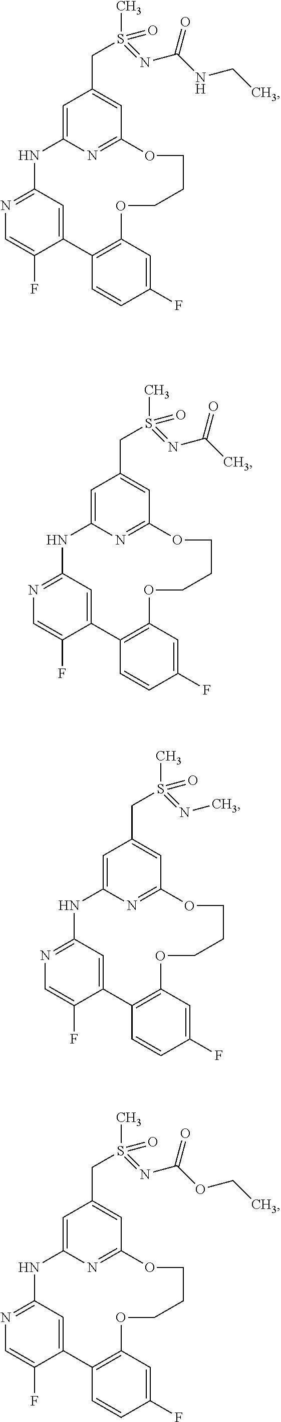 [SMILES: CCNC(=O)N=S(C)(=O)Cc1cc2nc(c1)OCCCOc1cc(F)ccc1-c1cc(ncc1F)N2 ; CC(=O)N=S(C)(=O)Cc1cc2nc(c1)OCCCOc1cc(F)ccc1-c1cc(ncc1F)N2 ; CN=S(C)(=O)Cc1cc2nc(c1)OCCCOc1cc(F)ccc1-c1cc(ncc1F)N2 ; CCOC(=O)N=S(C)(=O)Cc1cc2nc(c1)OCCCOc1cc(F)ccc1-c1cc(ncc1F)N2]

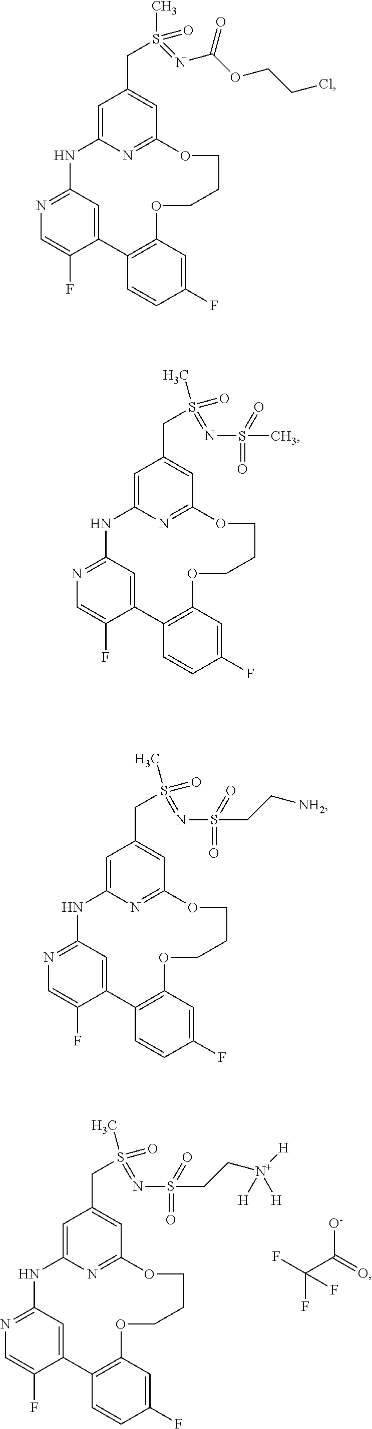 [SMILES: CS(=O)(Cc1cc2nc(c1)OCCCOc1cc(F)ccc1-c1cc(ncc1F)N2)=NC(=O)OCCCl ; CS(=O)(=O)N=S(C)(=O)Cc1cc2nc(c1)OCCCOc1cc(F)ccc1-c1cc(ncc1F)N2 ; CS(=O)(Cc1cc2nc(c1)OCCCOc1cc(F)ccc1-c1cc(ncc1F)N2)=NS(=O)(=O)CCN ; CS(=O)(Cc1cc2nc(c1)OCCCOc1cc(F)ccc1-c1cc(ncc1F)N2)=NS(=O)(=O)CC[NH3+] ; O=C([O-])C(F)(F)F]

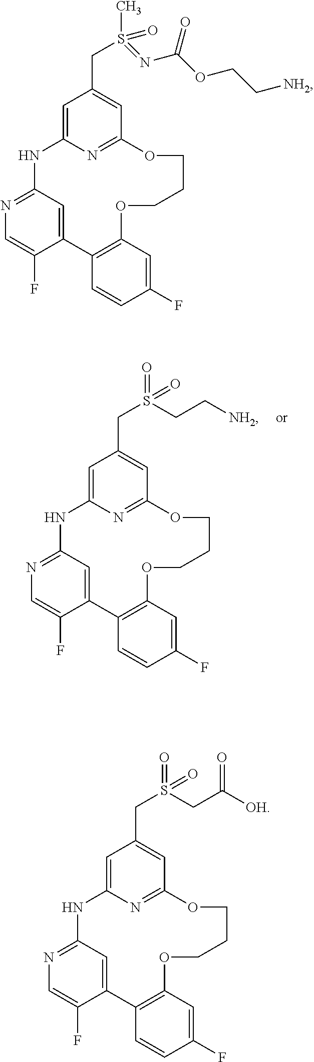 [SMILES: CS(=O)(Cc1cc2nc(c1)OCCCOc1cc(F)ccc1-c1cc(ncc1F)N2)=NC(=O)OCCN ; NCCS(=O)(=O)Cc1cc2nc(c1)OCCCOc1cc(F)ccc1-c1cc(ncc1F)N2 ; O=C(O)CS(=O)(=O)Cc1cc2nc(c1)OCCCOc1cc(F)ccc1-c1cc(ncc1F)N2]

Disclosed herein is a selective CDK9 inhibitor that is a compound of Formula (II):

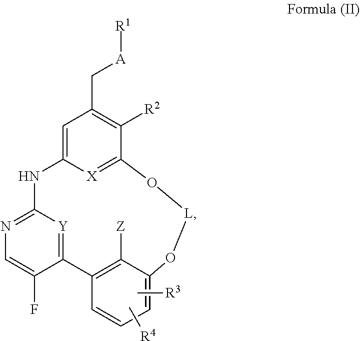

wherein
A is a bivalent moiety selected from the group consisting of —S—, —S(═O)—, —S(═O)₂—, —S(═O)(═NR⁵)—, or —S(═NR⁶)(═NR⁷)—;
Z is a hydrogen atom or a fluorine atom;
L is a C₃-C₄-alkylene moiety, wherein said moiety is optionally substituted with
   (i) one substituent selected from hydroxy, —NRR⁸R⁹, C₂-C₃-alkenyl-, C₂-C₃-alkynyl-, C₃-C₄-cycloalkyl-, hydroxy-C₁-C₃-alkyl, —(CH₂)NR⁸R⁹, and/or
   (ii) one or two or three or four substituents, identically or differently, selected from halogen and C₁-C₃-alkyl-,
or wherein one carbon atom of said C₃-C₈-alkylene moiety forms a three- or four-membered ring together with a bivalent moiety to which it is attached, wherein said bivalent moiety is selected from —CH₂CH₂—, —CH₂CH₂CH₂—, —CH₂OCH₂—;
X, Y are CH or N with the proviso that one of X and Y represents CH and one of X and Y represents N;
R¹ is C₁-C₆-alkyl-, C₃-C₇-alkenyl-, C₃-C₇-cycloalkyl-, or heterocyclyl-, wherein said group is optionally substituted with one or two or three substituents, identically or differently, selected from the group consisting of hydroxy, cyano, halogen, C₁-C₆-alkyl-, halo-C₁-C₃-alkyl-, C₁-C₆-alkoxy-, C₁-C₃-fluoroalkoxy-, —NH₂, alkylamino-, dialkylamino-, acetylamino-, N-methyl-N-acetylamino-, cyclic amines, —OP(═O)(OH)2, —C(═O)OH, and —C(═O)NH₂;
R² is a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R³, R⁴ are, independently from each other, a group selected from a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R⁵ is a hydrogen atom, cyano, —C(═O)R¹⁰, —C(═O)OR¹⁰, —S(═O₂) R¹⁰, —C(═O)NR⁸R⁹, C₁-C₆-alkyl-, C₃-C₇-cycloalkyl-, or heterocyclyl-, wherein said C₁-C₆-alkyl-, C₃-C₇-cycloalkyl-, heterocyclyl- group is optionally substituted with one, two or three substituents, identically or differently, selected from the group consisting of halogen, hydroxy, cyano, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, C₁-C₃-fluoroalkoxy-;
R⁶, R⁷ are, independently from each other, a hydrogen atom, cyano, —C(═O)R¹⁰, —C(═O)OR¹⁰, —S(═O)₂R¹⁰, —C(═O)NR⁸R⁹, C₁-C₆-alkyl-, C₃-C₇-cycloalkyl-, or heterocyclyl-, wherein said C₁-C₆-alkyl-, C₃-C₇-cycloalkyl-, heterocyclyl- group is optionally substituted with one, two or three substituents, identically or differently, selected from the group consisting of halogen, hydroxy, cyano, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, C₁-C₃-fluoroalkoxy-;
R⁸, R⁹ are, independently from each other, a hydrogen atom, C₁-C₆-alkyl-, C₃-C₇-cycloalkyl-, heterocyclyl-, phenyl-, benzyl-, or heteroaryl-, wherein said C₁-C₆-alkyl-, C₃-C₇-cycloalkyl-, heterocyclyl-, phenyl-, benzyl- or heteroaryl- group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-, or
R⁸ and R⁹, together with the nitrogen atom they are attached to, form a cyclic amine
R¹⁰ is C₁-C₆-alkyl-, halo-C₁-C₃-alkyl-, C₃-C₇-cycloalkyl-, heterocyclyl-, phenyl-, benzyl-, or heteroaryl-, wherein said group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-,
or the enantiomers, diastereomers, salts, solvates or salts of solvates thereof.

In some embodiments, the compound of Formula (II), or an enantiomer, diastereomer, salt, solvate or salt of solvate thereof is:

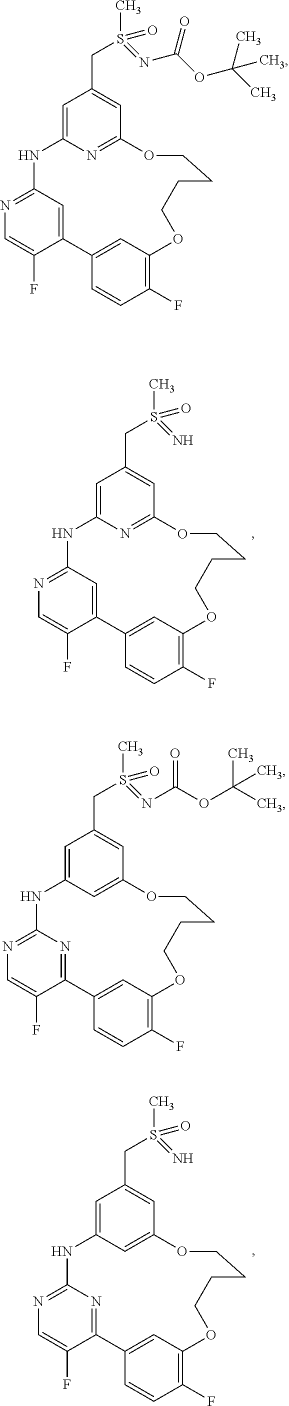 [SMILES: CC(C)(C)OC(=O)N=S(C)(=O)Cc1cc2nc(c1)OCCCCOc1cc(ccc1F)-c1cc(ncc1F)N2 ; CS(=N)(=O)Cc1cc2nc(c1)OCCCCOc1cc(ccc1F)-c1cc(ncc1F)N2 ; CC(C)(C)OC(=O)N=S(C)(=O)Cc1cc2cc(c1)OCCCCOc1cc(ccc1F)-c1nc(ncc1F)N2 ; CS(=N)(=O)Cc1cc2cc(c1)OCCCCOc1cc(ccc1F)-c1nc(ncc1F)N2]

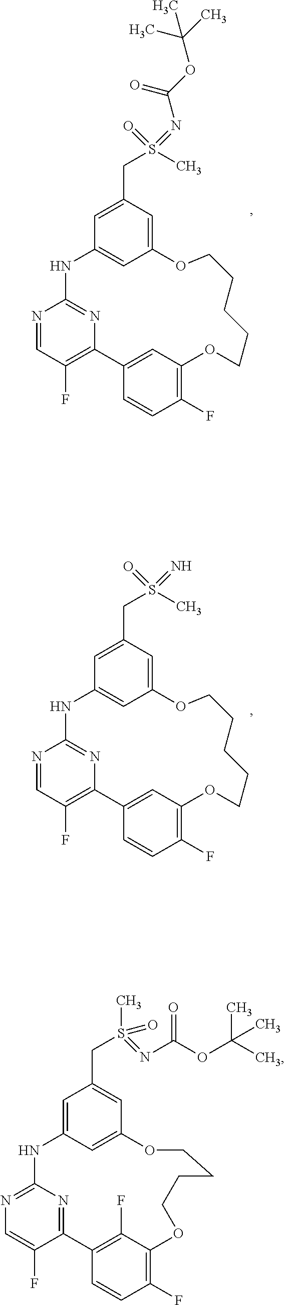 [SMILES: CC(C)(C)OC(=O)N=S(C)(=O)Cc1cc2cc(c1)OCCCCCOc1cc(ccc1F)-c1nc(ncc1F)N2 ; CS(=N)(=O)Cc1cc2cc(c1)OCCCCCOc1cc(ccc1F)-c1nc(ncc1F)N2 ; CC(C)(C)OC(=O)N=S(C)(=O)Cc1cc2cc(c1)OCCCCOc1c(F)ccc(c1F)-c1nc(ncc1F)N2]

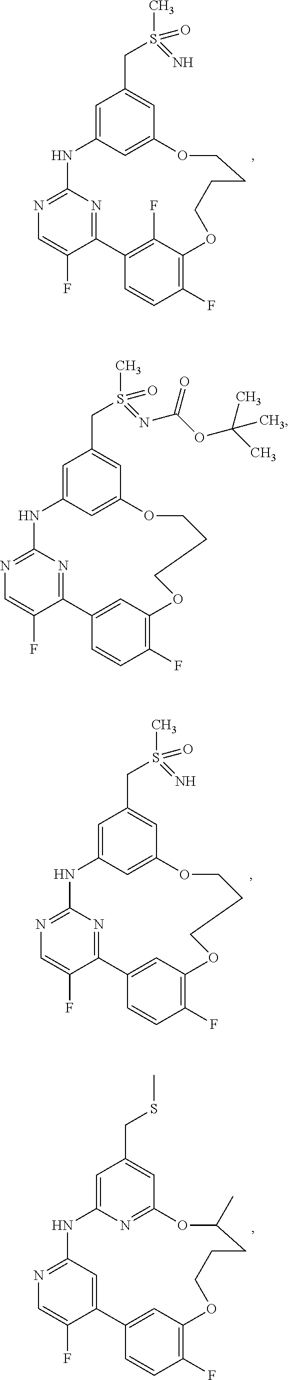 [SMILES: CS(=N)(=O)Cc1cc2cc(c1)OCCCCOc1c(F)ccc(c1F)-c1nc(ncc1F)N2 ; CC(C)(C)OC(=O)N=S(C)(=O)Cc1cc2cc(c1)OCCCOc1cc(ccc1F)-c1nc(ncc1F)N2 ; CS(=N)(=O)Cc1cc2cc(c1)OCCCOc1cc(ccc1F)-c1nc(ncc1F)N2 ; CSCc1cc2nc(c1)OC(C)CCCOc1cc(ccc1F)-c1cc(ncc1F)N2]

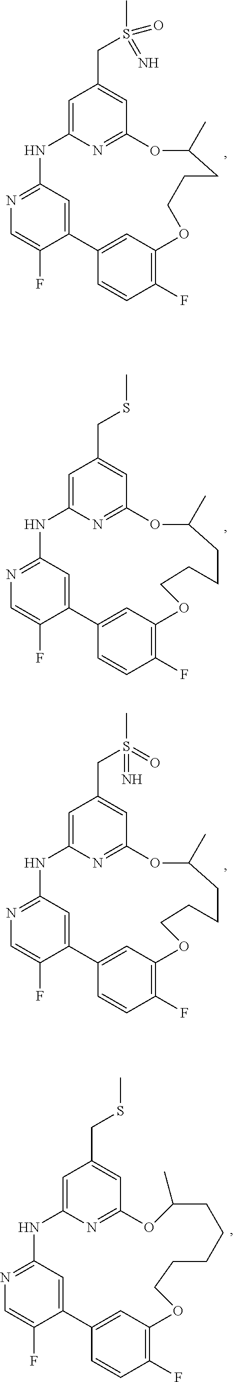

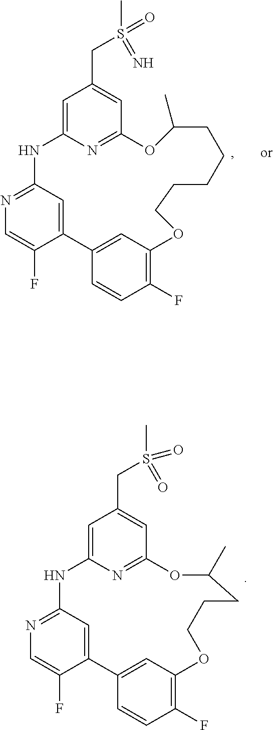 [SMILES: CC1CCCCCOc2cc(ccc2F)-c2cc(ncc2F)Nc2cc(CS(C)(=N)=O)cc(n2)O1 ; CC1CCCOc2cc(ccc2F)-c2cc(ncc2F)Nc2cc(CS(C)(=O)=O)cc(n2)O1]

Disclosed herein is a selective CDK9 inhibitor that is a compound of Formula (III):

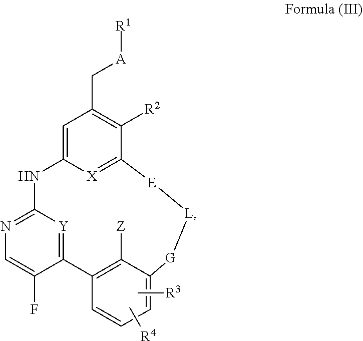

wherein
A is a bivalent moiety selected from the group consisting of —S—, —S(═O)—, —S(═O)₂—, —S(═O)(═NR⁵)—, or —S(═NR⁶)(═NR⁷)—;
G, E are, independently from each other, a bivalent moiety selected from the group consisting of —O—, —N(R^{A})—, —CH₂—, —CH(C₁-C₆-alkyl)-, —C(C₁-C₆-alkyl)-, and —S—, with the proviso that at least one of said bivalent moieties G and E is different from —O—;
Z is a hydrogen atom or a fluorine atom;
L is a C₃-C₄-alkylene moiety, wherein said moiety is optionally substituted with
   (i) one substituent selected from hydroxy, —NR⁸R⁹, C₂-C₃-alkenyl-, C₂-C₃-alkynyl-, C₃-C₄-cycloalkyl-, hydroxy-C₁-C₃-alkyl, and —(CH₂)NR⁸R⁹, and/or
   (ii) one or two or three or four substituents, identically or differently, selected from halogen and C₁-C₃-alkyl-,
or wherein one carbon atom of said C₃-C₅-alkylene moiety forms a three- or four-membered ring together with a bivalent moiety to which it is attached, wherein said bivalent moiety is —CH₂CH₂—, —CH₂CH₂CH₂—, or —CH₂OCH₂—;
X, Y are CH or N with the proviso that one of X and Y represents CH and one of X and Y represents N;
R¹ is C₁-C₆-alkyl-, C₃-C₇-alkenyl-, C₃-C₇-cycloalkyl-, or heterocyclyl-, wherein said group is optionally substituted with one or two or three substituents, identically or differently, selected from the group consisting of hydroxy, cyano, halogen, C₁-C₆-alkyl-, halo-C₁-C₃-alkyl-, C₁-C₆-alkoxy-, C₁-C₃-fluoroalkoxy-, —NH₂, alkylamino-, dialkylamino-, acetylamino-, N-methyl-N-acetylamino-, cyclic amines, —OP(═O)(OH)₂, —C(═O)OH, and —C(═O)NH₂;
R² is a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R³, R⁴ are, independently from each other, a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R⁵ is a hydrogen atom, cyano, —C(═O)R¹⁰, —C(═O)OR¹⁰, —S(═O₂) R¹⁰, —C(═O)NR⁸R⁹, C₁-C₆-alkyl-, C₃-C₇-cycloalkyl-, heterocyclyl-, wherein said C₁-C₆-alkyl-, C₃-C₇-cycloalkyl- and heterocyclyl- group is optionally substituted with one, two or three substituents, identically or differently, selected from the group consisting of halogen, hydroxy, cyano, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-;
R⁶, R⁷ are, independently from each other, a hydrogen atom, cyano, —C(═O)R¹⁰, —C(═O)OR¹⁰, —S(═O)₂R¹⁰, —C(═O)NR⁸R⁹, C₁-C₆-alkyl-, C₃-C₇-cycloalkyl-, or heterocyclyl-, wherein said C₁-C₆-alkyl-, C₃—C,-cycloalkyl- or heterocyclyl- group is optionally substituted with one, two or three substituents, identically or differently, selected from the group consisting of halogen, hydroxy, cyano, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-;
R⁸, R⁹ are, independently from each other, a hydrogen atom, C₁-C₆-alkyl-, C₃-C₇-cycloalkyl-, heterocyclyl-, phenyl-, benzyl-, or heteroaryl-, wherein said C₁-C₆-alkyl-, C₃-C₇-cycloalkyl-, heterocyclyl-, phenyl-, benzyl- or heteroaryl- group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-, or
R⁸ and R⁹, together with the nitrogen atom they are attached to, form a cyclic amine;
R¹⁰ is C₁-C₆-alkyl-, halo-C₁-C₃-alkyl-, C₃-C₇-cycloalkyl-, heterocyclyl-, phenyl-, benzyl-, or heteroaryl-, wherein said group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-;
R^{A} is a hydrogen atom or a C₁-C₆-alkyl- group,
or the enantiomers, diastereomers, salts, solvates or salts of solvates thereof.

In some embodiments, the compound of Formula (III), or an enantiomer, diastereomer, salt, solvate or salt of solvate thereof is:

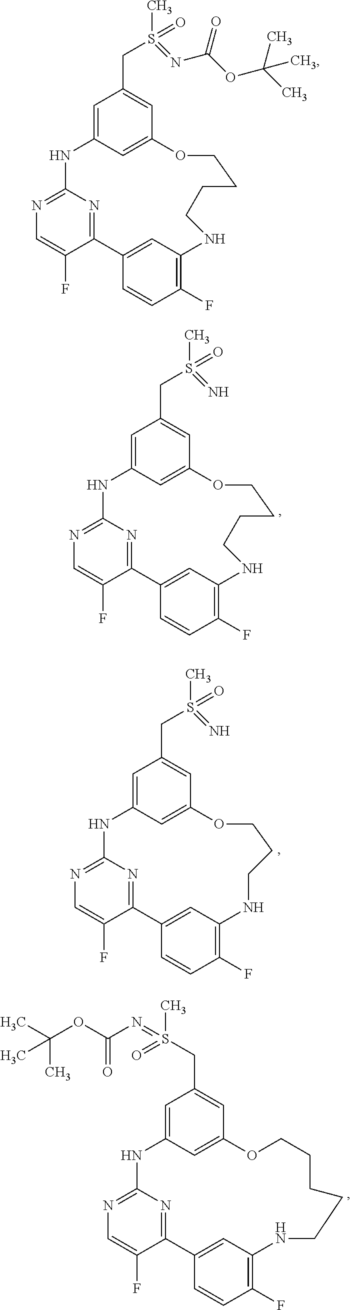

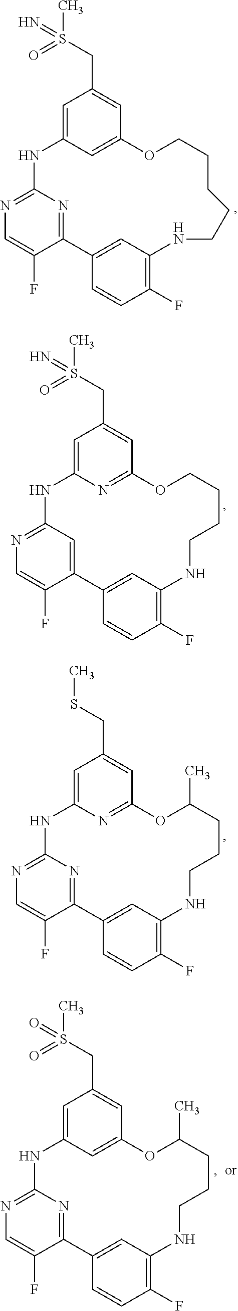 [SMILES: CS(=N)(=O)Cc1cc2cc(c1)OCCCCCNc1cc(ccc1F)-c1nc(ncc1F)N2 ; CS(=N)(=O)Cc1cc2nc(c1)OCCCCNc1cc(ccc1F)-c1cc(ncc1F)N2 ; CSCc1cc2nc(c1)OC(C)CCCNc1cc(ccc1F)-c1nc(ncc1F)N2 ; CC1CCCNc2cc(ccc2F)-c2nc(ncc2F)Nc2cc(CS(C)(=O)=O)cc(c2)O1]

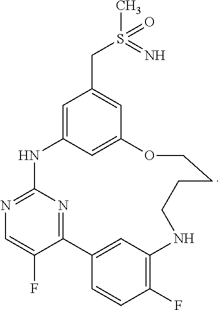 [SMILES: CS(=N)(=O)Cc1cc2cc(c1)OCCCCNc1cc(ccc1F)-c1nc(ncc1F)N2]

Disclosed herein is a selective CDK9 inhibitor that is a compound of Formula (IV):

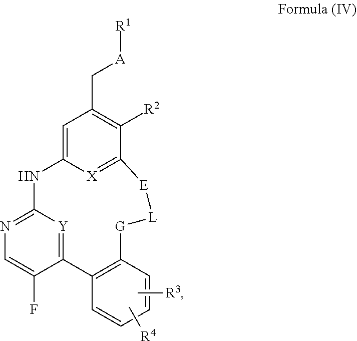

wherein
A is a bivalent moiety —S—, —S(═O)—, —S(═O)₂, or —S(═O)(N⁵)—;
G, E are, independently from each other, a bivalent moiety selected from the group consisting of —O—, —N(R^{A})—, —CH₂—, —CH(C₁-C₆-alkyl)-, —C(C₁-C₆-alkyl)-, —S—, —S(═O)—, and —S(═O)—, with the proviso that at least one of said bivalent moieties G and E is different from —O—;
L is a C₂-C₈-alkylene moiety, wherein said moiety is optionally substituted with
   (i) one substituent selected from hydroxy, —NRR⁷, C₂-C₃-alkenyl-, C₂-C₃-alkynyl-, C₃-C₄-cycloalkyl-, hydroxy-C₁-C₃-alkyl, —(CH₂)NR⁶R⁷, and/or
   (ii) one or two or three or four substituents, identically or differently, selected from halogen and C₁-C₃-alkyl-,
or wherein one carbon atom of said C₂-C₅-alkylene moiety forms a three- or four-membered ring together with a bivalent moiety to which it is attached, wherein said bivalent moiety is —CH₂CH₂—, —CH₂CH₂CH₂—, or —CH₂OCH₂—;
X, Y are CH or N with the proviso that one of X and Y represents CH and one of X and Y represents N;
R¹ is C₁-C₆-alkyl-, C₃-C₆-alkenyl-, C₃-C₇-alkynyl-, C₃-C₇-cycloalkyl-, heterocyclyl-, phenyl-, heteroaryl-, phenyl-C₁-C₃-alkyl-, or heteroaryl-C₁-C₃-alkyl-, wherein said group is optionally substituted with one or two or three substituents, identically or differently, selected from the group consisting of hydroxy, cyano, halogen, C₁-C₆-alkyl-, halo-C₁-C₃-alkyl-, C₁-C₆-alkoxy-, C₁-C₃-fluoroalkoxy-, —NH₂, alkylamino-, dialkylamino-, acetylamino-, N-methyl-N-acetylamino-, cyclic amines, —OP(═O)(OH)₂, —C(═O)OH, and —C(═O)NH₂;
R² is a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R³, R⁴ are, independently from each other, a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R⁵ is a hydrogen atom, cyano, —C(═O)R⁸, —C(═O)OR⁸, —S(═O)₂R⁸, —C(═O)NR⁸R⁷, C₁-C₆-alkyl-, C₃-C₇-cycloalkyl-, heterocyclyl-, phenyl-, or heteroaryl-, wherein said C₁-C₆-alkyl-, C₃-C₇-cycloalkyl-, heterocyclyl-, phenyl- or heteroaryl- group is optionally substituted with one, two or three substituents, identically or differently, selected from the group consisting of halogen, hydroxy, cyano, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-;
R⁶, R⁷ are, independently from each other, a hydrogen atom, C₁-C₆-alkyl-, C₃-C₇-cycloalkyl-, heterocyclyl-, phenyl-, benzyl- and heteroaryl-, wherein said C₁-C₆-alkyl-, C₃-C₇-cycloalkyl-, heterocyclyl-, phenyl-, benzyl- or heteroaryl- group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-, or
R⁶ and R⁷, together with the nitrogen atom they are attached to, form a cyclic amine;
R⁸ is C₁-C₆-alkyl-, halo-C₁-C₃-alkyl-, C₃-C₇-cycloalkyl-, heterocyclyl-, phenyl-, benzyl-, or heteroaryl-, wherein said group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-,
R^{A} is a hydrogen atom or a C₁-C₆-alkyl- group,
or the enantiomers, diastereomers, salts, solvates or salts of solvates thereof.

In some embodiments, the compound of Formula (IV), or an enantiomer, diastereomer, salt, solvate or salt of solvate thereof is:

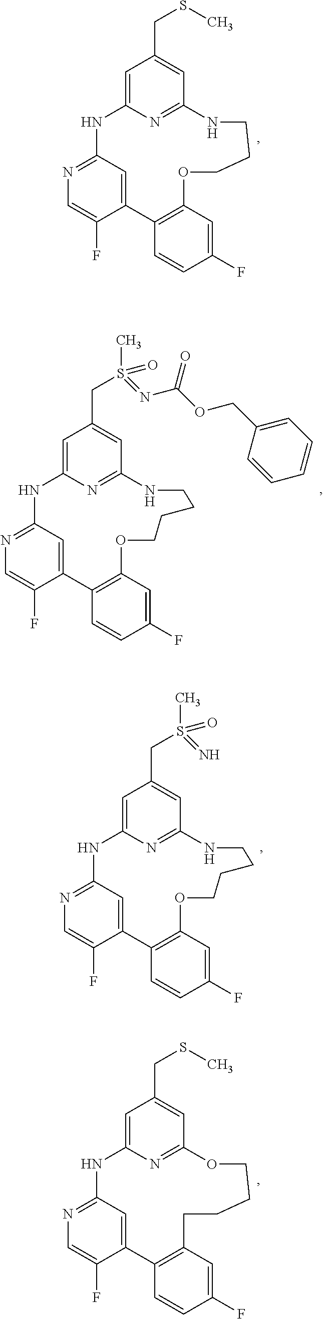 [SMILES: CSCc1cc2nc(c1)Nc1cc(c(F)cn1)-c1ccc(F)cc1OCCCN2 ; CS(=O)(Cc1cc2nc(c1)Nc1cc(c(F)cn1)-c1ccc(F)cc1OCCCCN2)=NC(=O)OCc1ccccc1 ; CS(=N)(=O)Cc1cc2nc(c1)Nc1cc(c(F)cn1)-c1ccc(F)cc1OCCCCN2 ; CSCc1cc2nc(c1)OCCCCc1cc(F)ccc1-c1cc(ncc1F)N2]

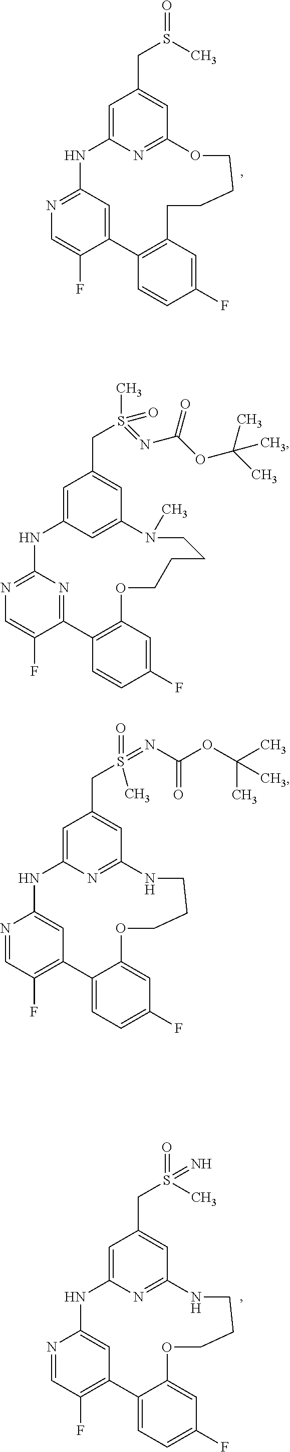

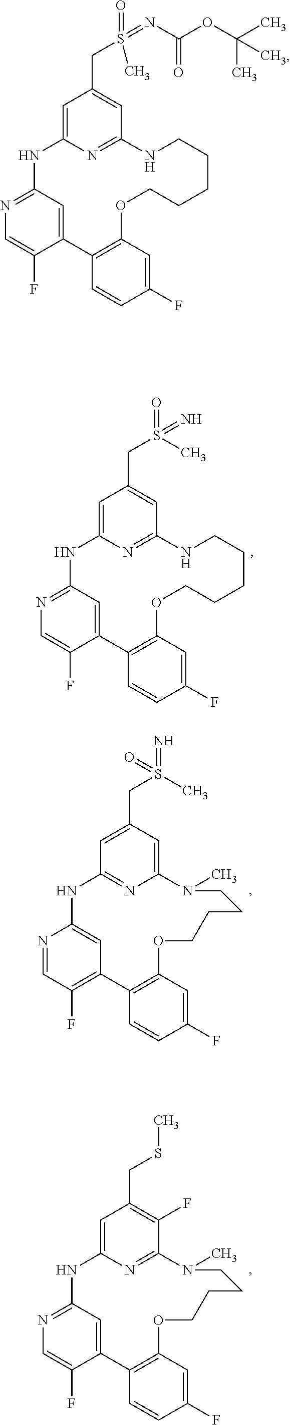

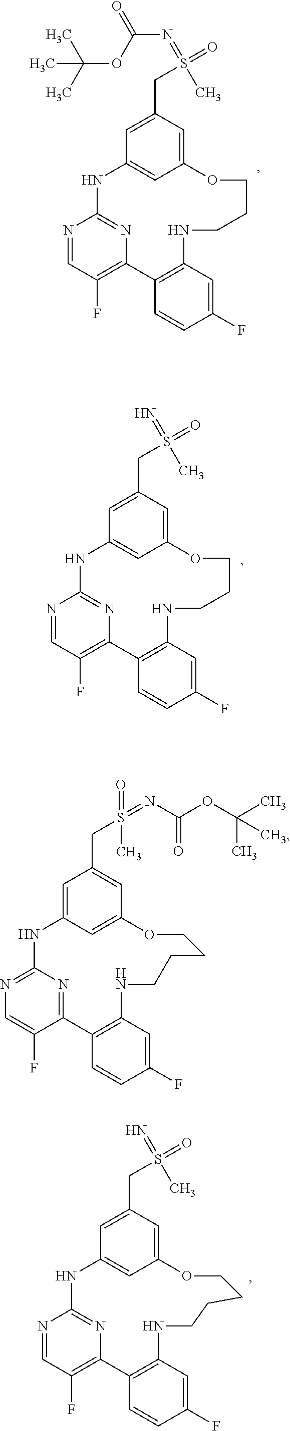 [SMILES: CC(C)(C)OC(=O)N=S(C)(=O)Cc1cc2cc(c1)OCCCNc1cc(F)ccc1-c1nc(ncc1F)N2 ; CS(=N)(=O)Cc1cc2cc(c1)OCCCNc1cc(F)ccc1-c1nc(ncc1F)N2 ; CC(C)(C)OC(=O)N=S(C)(=O)Cc1cc2cc(c1)OCCCCNc1cc(F)ccc1-c1nc(ncc1F)N2 ; CS(=N)(=O)Cc1cc2cc(c1)OCCCCNc1cc(F)ccc1-c1nc(ncc1F)N2]

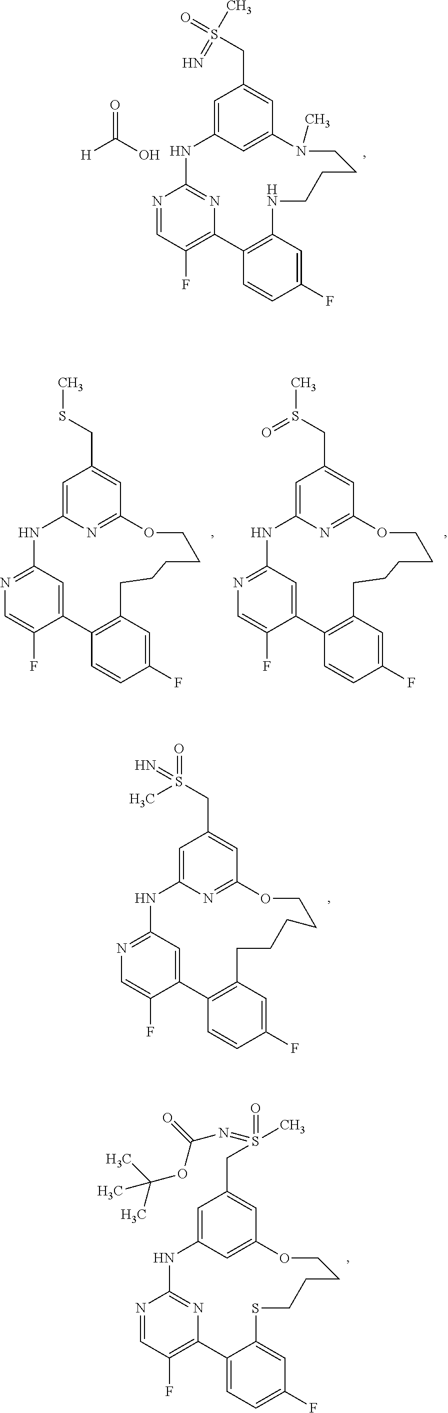

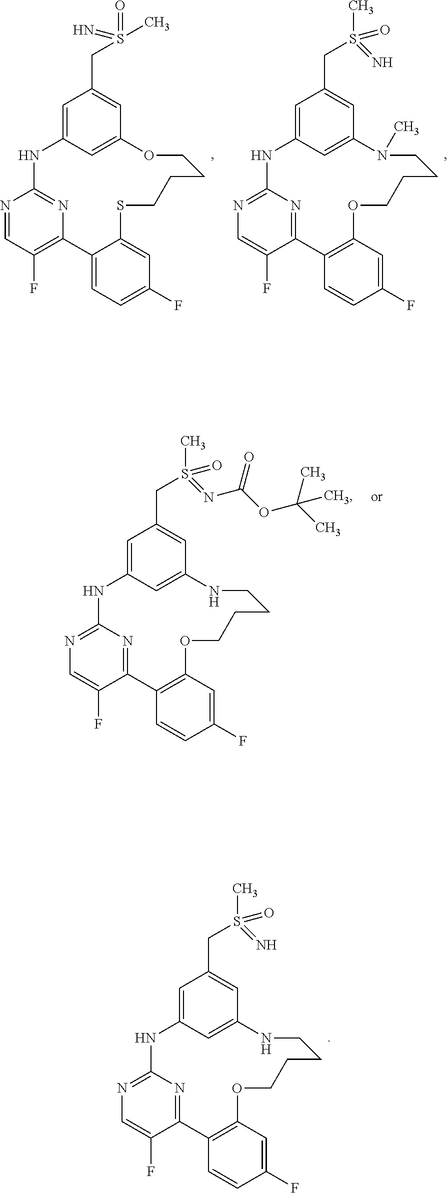

Disclosed herein is a selective CDK9 inhibitor that is a compound of Formula (V):

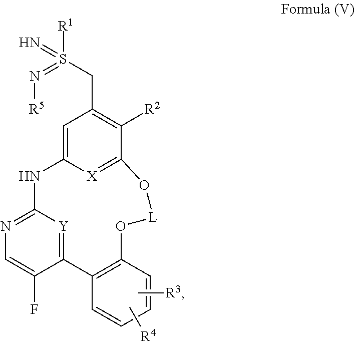

wherein
L is a C₂-C₈-alkylene group, wherein said group is optionally substituted with
   (i) one substituent selected from hydroxy, —NR⁸R⁷, C₂-C₃-alkenyl-, C₂-C₃-alkynyl-, C₃-C₄-cycloalkyl-, hydroxy-C₁-C₃-alkyl-, and —(CH₂)NR⁸R⁷, and/or
   (ii) one or two or three or four substituents, identically or differently, selected from halogen and C₁-C₃-alkyl-,
with the proviso that a C₂-alkylene group is not substituted with a hydroxy or a —NRR⁷ group, or wherein one carbon atom of said C₂-C₈-alkylene group forms a three- or four-membered ring together with a bivalent group to which it is attached, wherein said bivalent group is —CH₂CH₂—, —CH₂CH₂CH₂—, or —CH₂OCH₂—; X, Y are CH or N with the proviso that one of X and Y represents CH and one of X and Y represents N;
R¹ is C₁-C₆-alkyl-, C₃-C₆-alkenyl-, C₃-C₆-alkynyl-, C₃-C₇-cycloalkyl-, heterocyclyl-, phenyl-, heteroaryl-, phenyl-C₁-C₃-alkyl-, or heteroaryl-C₁-C₃-alkyl-, wherein said group is optionally substituted with one or two or three substituents, identically or differently, selected from the group consisting of hydroxy, cyano, halogen, C₁-C₆-alkyl-, halo-C₁-C₃-alkyl-, C₁-C₆-alkoxy-, C₁-C₃-fluoroalkoxy-, —NH₂, alkylamino-, dialkylamino-, acetylamino-, N-methyl-N-acetylamino-, cyclic amines, —OP(═O)(OH)₂, —C(═O)OH, and —C(═O)NH₂;
R² is a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R³, R are, independently from each other, a hydrogen atom, a fluorine atom, a chlorine atom, a bromine atom, cyano, C₁-C₃-alkyl-, C₁-C₃-alkoxy-, halo-C₁-C₃-alkyl-, or C₁-C₃-fluoroalkoxy-;
R⁵ is a hydrogen atom, cyano, —C(═O)R⁸, —C(═O)OR⁸, —S(═O)₂R⁸, —C(═O)NR⁸R⁷, C₁-C₆-alkyl-, C₃-C₇-cycloalkyl-, heterocyclyl-, phenyl-, or heteroaryl-, wherein said C₁-C₆-alkyl-, C₃-C₇-cycloalkyl-, heterocyclyl-, phenyl- or heteroaryl- group is optionally substituted with one, two or three substituents, identically or differently, selected from the group consisting of halogen, hydroxy, cyano, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-;
R⁶, R⁷ are, independently from each other a hydrogen atom, C₁-C₆-alkyl-, C₃-C₇-cycloalkyl-, heterocyclyl-, phenyl-, benzyl-, or heteroaryl-, wherein said C₁-C₆-alkyl-, C₃-C₇-cycloalkyl-, heterocyclyl-, phenyl-, benzyl- or heteroaryl- group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-, or
R⁶ and R⁷, together with the nitrogen atom they are attached to, form a cyclic amine;
R⁸ is C₁-C₆-alkyl-, halo-C₁-C₃-alkyl-, C₃-C₇-cycloalkyl-, heterocyclyl-, phenyl-, benzyl-, or heteroaryl-, wherein said group is optionally substituted with one, two or three substituents, identically or differently, selected from the group consisting of halogen, hydroxy, C₁-C₃-alkyl-, C₁-C₃-alkoxy-, —NH₂, alkylamino-, dialkylamino-, acetylamino-, N-methyl-N-acetylamino-, cyclic amines, halo-C₁-C₃-alkyl-, and C₁-C₃-fluoroalkoxy-,
or the enantiomers, diastereomers, salts, solvates or salts of solvates thereof.

In some embodiments, the compound of Formula (IV), or an enantiomer, diastereomer, salt, solvate or salt of solvate thereof is:

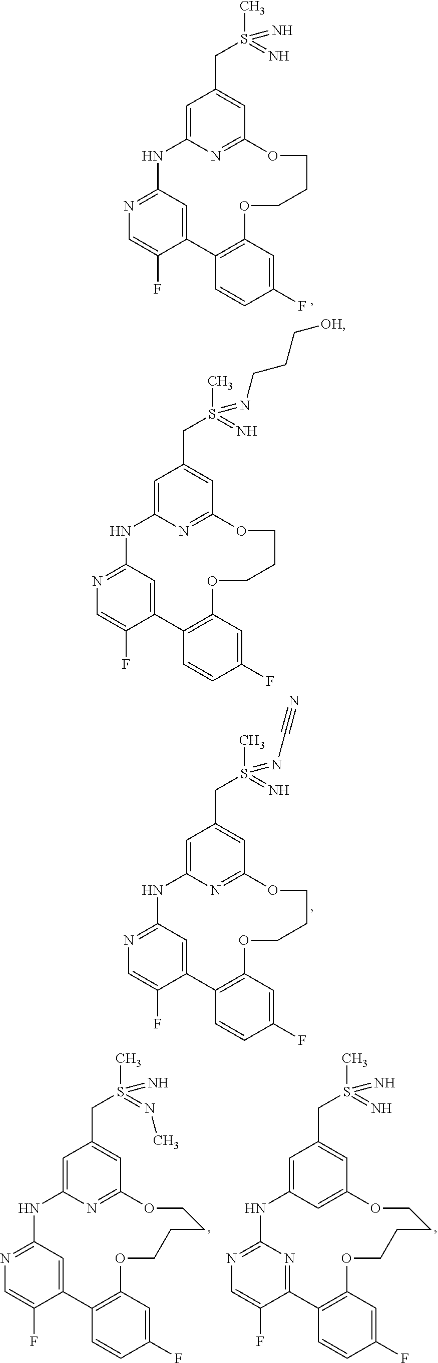

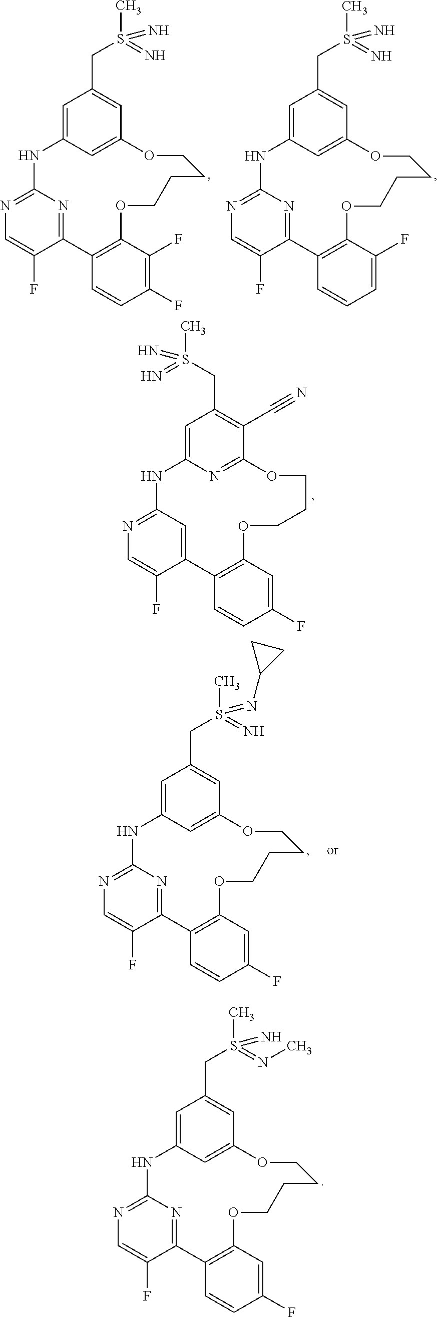

### Methods

Disclosed herein is a method of treating RAS mutant cancers with a selective CDK9 inhibitor.

Disclosed herein is a method of inhibiting RAS-mediated cell signaling comprising contacting a cell with an effective amount of a selective CDK9 inhibitor. Inhibition of RAS-mediated signal transduction can be assessed and demonstrated by a wide variety of ways known in the art. Non-limiting examples include a showing of (a) a decrease in GTPase activity of RAS; (b) a decrease in GTP binding affinity or an increase in GDP binding affinity; (c) an increase in K off of GTP or a decrease in K off of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf Kits and commercially available assays can be utilized for determining one or more of the above.

Also disclosed herein is a method of using a selective CDK9 inhibitor to treat disease conditions, including but not limited to conditions implicated by G12C KRAS, HRAS or NRAS mutation (e.g., cancer).

Disclosed herein is a method of treating a cancer that expresses a mutant form of a RAS protein in a subject in need thereof, the method comprising administering to the subject in need a therapeutically-effective amount of a selective CDK9 inhibitor.

Disclosed herein is a method of treating a cancer that expresses a mutant form of a RAS protein in a subject in need thereof, the method comprising administering to the subject in need a therapeutically-effective amount of a compound that is 5-fluoro-4-(4-fluoro-2-methoxyphenyl)-N-{4-[(S-methylsulfonimidoyl)methyl]pyridin-2-yl}pyridin-2-amine:

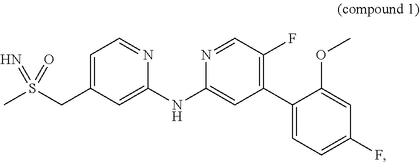 [SMILES: COc1cc(F)ccc1-c1cc(Nc2cc(CS(C)(=N)=O)ccn2)ncc1F]

or an enantiomer thereof, or an enantiomer thereof, or a pharmaceutically acceptable salt or solvate thereof.

Also disclosed herein is a method for treating cancer that expresses a mutant form of a RAS protein in a subject in need thereof with a selective CDK9 inhibitor, the method comprising:
(a) receiving a biological sample from the subject;
(b) assaying the biological sample for one or more RAS mutations;
(c) determining if one or more RAS mutations are present in the biological sample;
(d) employing the determination of one or more RAS mutations in the biological sample to predict responsiveness of the subject in need thereof to the compound, wherein the presence of one or more RAS mutations in the biological sample predicts that the subject will be responsive to the selective CDK9 inhibitor; and
(e) administering a therapeutically-effective amount of the selective CDK9 inhibitor to the subject.

Also disclosed herein is a method of selecting a subject with cancer for treatment with a selective CDK9 inhibitor, the method comprising:
(a) receiving a biological sample from the subject with cancer; and
(b) determining if one or more RAS mutations are present in the biological sample.

Also disclosed herein is a method for selecting a subject with cancer for treatment with a selective CDK9 inhibitor, the method comprising:
(a) receiving a biological sample from the subject;
(b) assaying the biological sample for one or more RAS mutations;
(c) determining if one or more RAS mutations are present in the biological sample;
(d) employing the determination of one or more RAS mutations in the biological sample to predict responsiveness of the subject in need thereof to the compound, wherein the presence of one or more RAS mutations in the biological sample predicts that the subject will be responsive to the selective CDK9 inhibitor; and
(e) administering a therapeutically-effective amount of the selective CDK9 inhibitor to the subject

Also disclosed herein is a method of predicting a therapeutic response for the treatment of cancer to a selective CDK9 inhibitor in a subject with cancer, the method comprising:
(a) determining if one or more RAS mutations are present in a biological sample obtained from the subject with cancer; and
(b) determining whether the subject is likely to respond administration of the selective CDK9 inhibitor.

Also disclosed herein is a method of contacting a cell with a selective CDK9 inhibitor, the method comprising:
(a) receiving a cell;
(b) assaying the cell for one or more RAS mutations;
(c) determining if one or more RAS mutations are present in the cell;
(d) employing the determination of one or more RAS mutations in the cell to predict responsiveness of the cell to the selective CDK9 inhibitor, wherein the presence of one or more RAS mutations in the cell predicts that the cell will be responsive to the selective CDK9 inhibitor; and
(e) selectively contacting a cell predicted to be responsive to the selective CDK9 inhibitor with the selective CDK9 inhibitor.

In some embodiments of a method of treating a cancer that expresses a mutant form of a RAS protein, the RAS protein is a mutant form of a NRAS protein. In some embodiments of a method of treating a cancer that expresses a mutant form of a RAS protein, the RAS protein is a mutant form of a HRAS protein. In some embodiments of a method of treating a cancer that expresses a mutant form of a RAS protein, the RAS protein is a mutant form of a KRAS protein.

In some embodiments of a method of treating a cancer that expresses a mutant form of a RAS protein, the mutant form of the RAS protein comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer that expresses a mutant form of a KRAS protein, the mutant form of the KRAS protein comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer that expresses a mutant form of a NRAS protein, the mutant form of the NRAS protein comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer that expresses a mutant form of a HRAS protein, the mutant form of the HRAS protein comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer that expresses a mutant form of a RAS protein, the mutant form of the RAS protein comprises one or more amino acid substitutions selected from the group consisting of G12C, G12D, G12V, G13S, and Q61R.

In some embodiments of a method of treating a cancer that expresses a mutant form of a RAS protein, the mutant form of the KRAS protein comprises one or more amino acid substitutions selected from the group consisting of G12C, G12D, G12V, G13S, and Q61R.

In some embodiments of a method of treating a cancer that expresses a mutant form of a RAS protein, the mutant form of the RAS protein comprises the amino acid substitution G12C.

In some embodiments of a method of treating a cancer that expresses a mutant form of a RAS protein, the mutant form of the RAS protein comprises the amino acid substitution G12D.

In some embodiments of a method of treating a cancer that expresses a mutant form of a RAS protein, the mutant form of the RAS protein comprises the amino acid substitution G12V.

In some embodiments of a method of treating a cancer that expresses a mutant form of a RAS protein, the mutant form of the RAS protein comprises the amino acid substitution G13S.

In some embodiments of a method of treating a cancer that expresses a mutant form of a RAS protein, the mutant form of the RAS protein comprises the amino acid substitution Q61R.

In some embodiments of a method of treating a cancer that expresses a mutant form of a RAS protein, the mutant form of the RAS protein comprises the amino acid substitution Q61H.

In some embodiments of a method of treating a cancer that expresses a mutant form of a KRAS protein, the mutant form of the KRAS protein comprises the amino acid substitution G12C.

In some embodiments of a method of treating a cancer that expresses a mutant form of a KRAS protein, the mutant form of the KRAS protein comprises the amino acid substitution G12D.

In some embodiments of a method of treating a cancer that expresses a mutant form of a KRAS protein, the mutant form of the KRAS protein comprises the amino acid substitution G12V.

In some embodiments of a method of treating a cancer that expresses a mutant form of a KRAS protein, the mutant form of the KRAS protein comprises the amino acid substitution G13S.

In some embodiments of a method of treating a cancer that expresses a mutant form of a KRAS protein, the mutant form of the KRAS protein comprises the amino acid substitution Q61R.

In some embodiments of a method of treating a cancer that expresses a mutant form of a KRAS protein, the mutant form of the KRAS protein comprises the amino acid substitution Q61H.

In some embodiments of a method of treating a cancer that expresses a mutant form of a NRAS protein, the mutant form of the NRAS protein comprises the amino acid substitution G12C.

In some embodiments of a method of treating a cancer that expresses a mutant form of a NRAS protein, the mutant form of the NRAS protein comprises the amino acid substitution G12D.

In some embodiments of a method of treating a cancer that expresses a mutant form of a NRAS protein, the mutant form of the NRAS protein comprises the amino acid substitution G12V.

In some embodiments of a method of treating a cancer that expresses a mutant form of a NRAS protein, the mutant form of the NRAS protein comprises the amino acid substitution G13S.

In some embodiments of a method of treating a cancer that expresses a mutant form of a NRAS protein, the mutant form of the NRAS protein comprises the amino acid substitution Q61R.

In some embodiments of a method of treating a cancer that expresses a mutant form of a NRAS protein, the mutant form of the NRAS protein comprises the amino acid substitution Q61H.

In some embodiments of a method of treating a cancer that expresses a mutant form of a HRAS protein, the mutant form of the HRAS protein comprises the amino acid substitution G12C.

In some embodiments of a method of treating a cancer that expresses a mutant form of a HRAS protein, the mutant form of the HRAS protein comprises the amino acid substitution G12D.

In some embodiments of a method of treating a cancer that expresses a mutant form of a HRAS protein, the mutant form of the HRAS protein comprises the amino acid substitution G12V.

In some embodiments of a method of treating a cancer that expresses a mutant form of a HRAS protein, the mutant form of the HRAS protein comprises the amino acid substitution G13S.

In some embodiments of a method of treating a cancer that expresses a mutant form of a HRAS protein, the mutant form of the HRAS protein comprises the amino acid substitution Q61R.

In some embodiments of a method of treating a cancer that expresses a mutant form of a HRAS protein, the mutant form of the HRAS protein comprises the amino acid substitution Q61H.

In some embodiments of a method of treating a cancer associated with a RAS mutation, the RAS mutation is a NRAS mutation.

In some embodiments of a method of treating a cancer associated with a RAS mutation, the RAS mutation is a HRAS mutation.

In some embodiments of a method of treating a cancer associated with a RAS mutation, the RAS mutation is a KRAS mutation.

In some embodiments of a method of treating a cancer associated with a RAS mutation, the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer associated with a KRAS mutation, the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer associated with a NRAS mutation, the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer associated with a HRAS mutation, the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

In some embodiments of a method of treating a cancer associated with a RAS mutation, the mutation comprises one or more amino acid substitutions selected from the group consisting of G12C, G12D, G12V, G13S, and Q61R.

In some embodiments of a method of treating a cancer associated with a RAS mutation, the mutation comprises the amino acid substitution G12S.

In some embodiments of a method of treating a cancer associated with a RAS mutation, the mutation comprises the amino acid substitution G12C.

In some embodiments of a method of treating a cancer associated with a RAS mutation, the mutation comprises the amino acid substitution G12D.

In some embodiments of a method of treating a cancer associated with a RAS mutation, the mutation comprises the amino acid substitution G12V.

In some embodiments of a method of treating a cancer associated with a RAS mutation, the mutation comprises the amino acid substitution G13S.

In some embodiments of a method of treating a cancer associated with a RAS mutation, the mutation comprises the amino acid substitution G13D.

In some embodiments of a method of treating a cancer associated with a RAS mutation, the mutation comprises the amino acid substitution Q61R.

In some embodiments of a method of treating a cancer associated with a RAS mutation, the mutation comprises the amino acid substitution Q61H.

Methods for detecting a mutation in a KRAS, HRAS or NRAS protein arm known by those of skill in the art. These methods include, but are not limited to, detection of a KRAS. HRAS or NRAS mutant using a binding agent (e.g, an antibody) specific for the mutant protein, protein electrophoresis and Western blotting, and direct peptide sequencing.

Determining whether a tumor or cancer comprises a G12C KRAS, HRAS or NRAS mutation can be undertaken by assessing the nucleotide sequence encoding the KRAS, HRAS or NRAS protein, by assessing the amino acid sequence of the KRAS, HRAS or NRAS protein, or by assessing the characteristics of a putative KRAS, HRAS or NRAS mutant protein. The sequence of wild-type human KRAS, HRAS or NRAS is known in the art, (e.g. Accession No. NP203524).

Methods for detecting a mutation in a KRAS, HRAS or NRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS, HRAS or NRAS mutations by real-time PCR. In real-time PCR, fluorescent probes specific for the KRAS, HRAS or NRAS G12C mutation are used. When a mutation is present, the probe binds and fluorescence is detected. In some embodiments, the KRAS, HRAS or NRAS G12C mutation is identified using a direct sequencing method of specific regions (e.g., exon 2 and/or exon 3) in the KRAS, HRAS or NRAS gene. This technique will identify all possible mutations in the region sequenced.

Methods for detecting a mutation in a KRAS, HRAS or NRAS protein are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing.

Methods for determining whether a tumor or cancer comprises a G12C KRAS, HRAS or NRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

In some embodiments of a method of treating a cancer that expresses a mutant form of a RAS protein or a cancer associated with a RAS mutation, the biological sample is a tumor biopsy. In some embodiments of a method of treating a cancer that expresses a mutant form of a RAS protein or a cancer associated with a RAS mutation, the biological sample is an aspirate.

In some embodiments of a method of treating a cancer that expresses a mutant form of a RAS protein or a cancer associated with a RAS mutation, the cancer is bladder cancer, brain cancer, brain metastases, breast cancer, cervical cancer, colorectal cancer, endometrial cancer, gastrointestinal cancer, glioblastoma, head and neck cancer, leukemias, liver cancer, lung cancer, lymphomas, melanoma, multiple myeloma, ovarian cancer, pancreatic cancer, peritoneal cancer, prostate cancer, renal cancer, salivary gland carcinoma, thyroid cancer, or uterine carcinoma.

In some embodiments of a method of treating a cancer that expresses a mutant form of a RAS protein or a cancer associated with a RAS mutation, the cancer is non-small cell lung cancer, pancreatic cancer, or glioblastoma.

In some embodiments of a method of treating a cancer that expresses a mutant form of a RAS protein or a cancer associated with a RAS mutation, the cancer is colorectal cancer, non-small cell lung cancer, or pancreatic cancer.

In some embodiments of a method of treating a cancer that expresses a mutant form of a RAS protein or a cancer associated with a RAS mutation, the cancer is glioblastoma.

In some embodiments of a method of treating a cancer that expresses a mutant form of a RAS protein or a cancer associated with a RAS mutation, the cancer is colorectal cancer.

In some embodiments of a method of treating a cancer that expresses a mutant form of a RAS protein or a cancer associated with a RAS mutation, the cancer is non-small cell lung cancer.

In some embodiments of a method of treating a cancer that expresses a mutant form of a RAS protein or a cancer associated with a RAS mutation, the cancer is pancreatic cancer.

In some embodiments of a method of treating a cancer that expresses a mutant form of a RAS protein or a cancer associated with a RAS mutation, the cancer is Cardiac sarcoma (angiosarcoma, fibrosarcoma, rhabdomyosarcoma, liposarcoma), myxoma, rhabdomyomam, fibroma, lipoma and teratoma; Lung, bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, liporma, neurofibroma, fibroma), large bowel (adenocarcinoma, tubular adenoma, villous adenoma, hamartoma, leiomyoma); Genitourinary tract kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcmoma, hepatoblastoma, angiosarcoma, hepatocellular adenoma, hemangioma; Biliary tract: gall bladder carcinoma, ampullary carcinoma, cholangiocarcinoma. Bone, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system; skull (osteoma, hemangioma, granuloma, xanthoma, osteitis deformans), meninges (meningioma, meningiosarcoma, gliomatosis), brain (astrocytoma, medulloblastoma, glioma, ependymoma, gemrinoma (pinealoma), glioblastoma multiform, oligodendroglioma, schwannoma, retinoblastoma, congenital tumors), spinal cord neurofibroma, meningioma, glioma, sarcoma); Gynecological; uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic, blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or an enantiomer thereof, or a pharmaceutically acceptable salt or solvate thereof, can be determined via established animal and human experimental protocols as well as in the examples described herein. For example, toxicity and therapeutic efficacy of compound 1, or an enantiomer thereof, or a pharmaceutically acceptable salt or solvate thereof, can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or an enantiomer thereof, or a pharmaceutically acceptable salt or solvate thereof, that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of between about 1 mg and about 50 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of between about 1 mg and about 30 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of between about 10 mg and about 30 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of between about 20 mg and about 30 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of about 5 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of about 10 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of about 15 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of about 20 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of about 25 mg.

In some embodiments of a method of treating a cancer that expresses a mutant form of a RAS protein or a cancer associated with a RAS mutation, the selective CDK9 inhibitor is compound 1, or an enantiomer thereof, or a pharmaceutically acceptable salt or solvate thereof, and is administered at a dose of about 30 mg.

### Administration

Administration of the selective CDK9 inhibitor (e.g., compound 1, or an enantiomer thereof, or a pharmaceutically acceptable salt or solvate thereof), is at a dosage described herein or at other dose levels and compositions determined and contemplated by a medical practitioner. In certain embodiments, the selective CDK9 inhibitor (e.g., compound 1, or an enantiomer thereof, or a pharmaceutically acceptable salt or solvate thereof), is administered for prophylactic and/or therapeutic treatments. In certain therapeutic applications, the selective CDK9 inhibitor (e.g., compound 1, or an enantiomer thereof, or a pharmaceutically acceptable salt or solvate thereof), is administered to a subject already suffering from a disease in an amount sufficient to cure the disease or at least partially arrest or ameliorate the symptoms. Amounts effective for this use depend on the age of the subject, severity of the disease, previous therapy, the subject's health status, weight, and response to the compositions, and the judgment of the treating physician. Therapeutically effective amounts are optionally determined by methods including, but not limited to, a dose escalation clinical trial.

In prophylactic applications, the compositions described herein are administered to a subject susceptible to or otherwise at risk of a particular disease, e.g., cancer. Such an amount is defined to be a “prophylactically effective amount or dose.” In this use, the precise amounts also depend on the subject's age, state of health, weight, and the like. When used in a subject, effective amounts for this use will depend on the risk or susceptibility of developing the particular disease, previous therapy, the subject's health status and response to the compositions, and the judgment of the treating physician.

In certain embodiments wherein the subject's condition does not improve, upon the doctor's discretion the administration of a composition described herein are administered chronically, that is, for an extended period of time, including throughout the duration of the subject's life in order to ameliorate or otherwise control or limit the symptoms of the subject's disease. In other embodiments, administration of a composition continues until complete or partial response of a disease.

In some embodiments of a method of treating a cancer that expresses a mutant form of a RAS protein or a cancer associated with a RAS mutation, the administration repeats until evidence of disease progression occurs.

In some embodiments, the selective CDK9 inhibitor (e.g., compound 1, or an enantiomer thereof, or a pharmaceutically acceptable salt or solvate thereof), is administered orally.

In some embodiments, the selective CDK9 inhibitor (e.g., compound 1, or an enantiomer thereof, or a pharmaceutically acceptable salt or solvate thereof), is administered intravenously.

In some embodiments, the selective CDK9 inhibitor (e.g., compound 1, or an enantiomer thereof, or a pharmaceutically acceptable salt or solvate thereof), is administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, the selective CDK9 inhibitor (e.g., compound 1, or an enantiomer thereof, or a pharmaceutically acceptable salt or solvate thereof), is administered to a subject who is in a fasted state for at least 8 hours. In other embodiments, compound 1, or a pharmaceutically acceptable salt thereof, is administered to a subject who is in a fasted state for at least 10 hours. In yet other embodiments, the selective CDK9 inhibitor (e.g., compound 1, or an enantiomer thereof, or a pharmaceutically acceptable salt or solvate thereof), is administered to a subject who is in a fasted state for at least 12 hours. In other embodiments, the selective CDK9 inhibitor (e.g., compound 1, or an enantiomer thereof, or a pharmaceutically acceptable salt or solvate thereof), is administered to a subject who has fasted overnight.

In other embodiments, the selective CDK9 inhibitor (e.g., compound 1, or an enantiomer thereof, or a pharmaceutically acceptable salt or solvate thereof), is administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, the selective CDK9 inhibitor (e.g., compound 1, or an enantiomer thereof, or a pharmaceutically acceptable salt or solvate thereof), is administered to a subject in a fed state 30 minutes post-meal. In other instances, the selective CDK9 inhibitor (e.g., compound 1, or an enantiomer thereof, or a pharmaceutically acceptable salt or solvate thereof), is administered to a subject in a fed state 1 hour post-meal. In yet further embodiments, compound 1, or an enantiomer thereof, or a pharmaceutically acceptable salt or solvate thereof, is administered to a subject with food.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks.

In some embodiments, a treatment cycle lasts five weeks. The number of treatment doses scheduled within each cycle also varies depending on the drugs being given.

In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered in 21-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for multiple 21-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least one 21-day cycle. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least two 21-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least three 21-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least four 21-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least five 21-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least six 21-day cycles.

In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered in 28-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for multiple 28-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least one 28-day cycle. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least two 28-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least three 28-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least four 28-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least five 28-day cycles. In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered for at least six 28-day cycles.

### Combination

Disclosed herein is a method of treating a cancer that expresses a mutant form of a RAS protein in subjects in need thereof, the method comprising administering to a subject in need a therapeutically-effective amount of a selective CDK9 inhibitor in combination with an additional therapeutic agent.

Also disclosed herein is a method of treating a cancer that expresses a mutant form of a RAS protein in a subject in need thereof, the method comprising administering to the subject in need a therapeutically-effective amount of a compound that is 5-fluoro-4-(4-fluoro-2-methoxyphenyl)-N-{4-[(S-methylsulfonimidoyl)methyl]pyridin-2-yl}pyridin-2-amine:

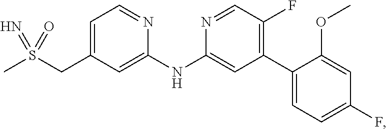 [SMILES: COc1cc(F)ccc1-c1cc(Nc2cc(CS(C)(=N)=O)ccn2)ncc1F]

or an enantiomer thereof, or a pharmaceutically acceptable salt or solvate thereof, in combination with an additional therapeutic agent.

In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), and the additional therapeutic agent are administered sequentially.

In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), and the additional therapeutic agent are administered simultaneously.

In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered before the additional therapeutic agent.

In some embodiments of a method of treating a cancer that expresses a mutant form of a RAS protein or a cancer associated with a RAS mutation, the selective CDK9 inhibitor (e.g., compound 1, or an enantiomer thereof, or a pharmaceutically acceptable salt or solvate thereof), is administered after the additional therapeutic agent.

In some embodiments of a method of treating a cancer that expresses a mutant form of a RAS protein or a cancer associated with a RAS mutation, the additional therapeutic agent is an AKT inhibitor, BCL2 inhibitor, BTK inhibitor, EGFR inhibitor, farnesyl transferase inhibitor, HER2 inhibitor, MEK inhibitor, mTOR inhibitor, PI3K inhibitor, RAF inhibitor, RAS inhibitor, or any combinations thereof.

In some embodiments of a method of treating a cancer that expresses a mutant form of a RAS protein or a cancer associated with a RAS mutation, the additional therapeutic agent is an KRAS inhibitor, such as e.g., AMG-510, MRTX849, and ARS-3248.

In some embodiments of a method of treating a cancer that expresses a mutant form of a RAS protein or a cancer associated with a RAS mutation, the additional therapeutic agent is an NRAS inhibitor.

In some embodiments of a method of treating a cancer that expresses a mutant form of a RAS protein or a cancer associated with a RAS mutation, the additional therapeutic agent is an HRAS inhibitor.

In some embodiments of a method of treating a cancer that expresses a mutant form of a RAS protein or a cancer associated with a RAS mutation, the compound and the additional therapeutic agent are administered sequentially.

In some embodiments of a method of treating a cancer that expresses a mutant form of a RAS protein or a cancer associated with a RAS mutation, the compound and the additional therapeutic agent are administered simultaneously.

In some embodiments of a method of treating a cancer that expresses a mutant form of a RAS protein or a cancer associated with a RAS mutation, the compound is administered before the additional therapeutic agent.

In some embodiments of a method of treating a cancer that expresses a mutant form of a RAS protein or a cancer associated with a RAS mutation, the compound is administered after the additional therapeutic agent.

In some embodiments of a method of treating a cancer that expresses a mutant form of a RAS protein or a cancer associated with a RAS mutation, the additional therapeutic agent is an anti-tumor agent such as alkylating agent, anti-metabolite, plant-derived anti-tumor agent, hormonal therapy agent, topoisomerase inhibitor, camptothecin derivative, kinase inhibitor, targeted drug, antibodies, interferon and/or biological response modifier, anti-angiogenic compound, or other anti-tumor drugs.

In some embodiments of a method of treating a cancer that expresses a mutant form of a RAS protein or a cancer associated with a RAS mutation, the additional therapeutic agent is an alkylating agent including, but are not limited to, nitrogen mustard N-oxide, cyclophosphamide, ifosfamide, thiotepa, ranimustine, nimustine, temozolomide, altretamine, apaziquone, brostallicin, bendamustine, carmustine, estramustine, fotemustine, glufosfamide, mafosfamide, bendamustin, and mitolactol; platinum-coordinated alkylating compounds include, but are not limited to, cisplatin, carboplatin, eptaplatin, lobaplatin, nedaplatin, oxaliplatin, and satraplatin.

In some embodiments of a method of treating a cancer that expresses a mutant form of a RAS protein or a cancer associated with a RAS mutation, the additional therapeutic agent is an anti-metabolite including, but are not limited to, methotrexate, 6-mercaptopurine riboside, mercaptopurine, 5-fluorouracil alone or in combination with leucovorin, tegafur, doxifluridine, carmofur, cytarabine, cytarabine ocfosfate, enocitabine, gemcitabine, fludarabin, 5-azacitidine, capecitabine, cladribine, clofarabine, decitabine, eflornithine, ethynylcytidine, cytosine arabinoside, hydroxyurea, melphalan, nelarabine, nolatrexed, ocfosfite, disodium premetrexed, pentostatin, pelitrexol, raltitrexed, triapine, trimetrexate, vidarabine, vincristine, and vinorelbine.

In some embodiments of a method of treating a cancer that expresses a mutant form of a RAS protein or a cancer associated with a RAS mutation, the additional therapeutic agent is a hormonal therapy agent including, but are not limited to, exemestane, Lupron, anastrozole, doxercalciferol, fadrozole, formestane, 11-beta hydroxysteroid dehydrogenase 1 inhibitors, 17-alpha hydroxylase/17,20 lyase inhibitors such as abiraterone acetate, 5-alpha reductase inhibitors such as finasteride and epristeride, anti-estrogens such as tamoxifen citrate and fulvestrant, Trelstar, toremifene, raloxifene, lasofoxifene, letrozole, anti-androgens such as bicalutamide, flutamide, mifepristone, nilutamide, and anti-progesterones and combinations thereof.

In some embodiments of a method of treating a cancer that expresses a mutant form of a RAS protein or a cancer associated with a RAS mutation, the additional therapeutic agent is a plant-derived anti-tumor substance including, e.g., those selected from mitotic inhibitors, for example epothilones such as sagopilone, ixabepilone, epothilone B, vinblastine, vinflunine, docetaxel, and paclitaxel.

In some embodiments of a method of treating a cancer that expresses a mutant form of a RAS protein or a cancer associated with a RAS mutation, the additional therapeutic agent is a cytotoxic topoisomerase inhibiting agent including, but are not limited to, aclarubicin, doxorubicin, amonafide, belotecan, camptothecin, 10-hydroxycamptothecin, 9-aminocamptothecin, diflomotecan, irinotecan, topotecan, edotecarin, epimbicin, etoposide, exatecan, gimatecan, lurtotecan, mitoxantrone, pirambicin, pixantrone, rubitecan, sobuzoxane, tafluposide, and combinations thereof.

In some embodiments of a method of treating a cancer that expresses a mutant form of a RAS protein or a cancer associated with a RAS mutation, the additional therapeutic agent is an immunological including interferons such as interferon alpha, interferon alpha-2a, interferon alpha-2b, interferon beta, interferon gamma-1a and interferon gamma-n1, and other immune enhancing agents such as L19-IL2 and other IL2 derivatives, filgrastim, lentinan, sizofilan, TheraCys, ubenimex, aldesleukin, alemtuzumab, BAM-002, dacarbazine, daclizumab, denileukin, gemtuzumab, ozogamicin, ibritumomab, imiquimod, lenograstim, lentinan, melanoma vaccine, molgramostim, sargramostim, tasonermin, tecleukin, thymalasin, tositumomab, Vimlizin, epratuzumab, mitumomab, oregovomab, pemtumomab, and sipuleucel-T.

In some embodiments of a method of treating a cancer that expresses a mutant form of a RAS protein or a cancer associated with a RAS mutation, the additional therapeutic agent is an anti-angiogenic compound including, but are not limited to, acitretin, aflibercept, angiostatin, aplidine, asentar, axitinib, recentin, bevacizumab, brivanib alaninat, cilengtide, combretastatin, DAST, endostatin, fenretinide, halofuginone, pazopanib, ranibizumab, rebimastat, removab, revlimid, sorafenib, vatalanib, squalamine, sunitinib, telatinib, thalidomide, ukrain, and vitaxin.

In some embodiments of a method of treating a cancer that expresses a mutant form of a RAS protein or a cancer associated with a RAS mutation, the additional therapeutic agent is an antibody including, but are not limited to, trastuzumab, cetuximab, bevacizumab, rituximab, ticilimumab, ipilimumab, lumiliximab, catumaxomab, atacicept, oregovomab, and alemtuzumab.

In some embodiments of a method of treating a cancer that expresses a mutant form of a RAS protein or a cancer associated with a RAS mutation, the additional therapeutic agent is a VEGF inhibitor such as, e.g., sorafenib, DAST, bevacizumab, sunitinib, recentin, axitinib, aflibercept, telatinib, brivanib alaninate, vatalanib, pazopanib, ranibizumab, and toceranib.

In some embodiments of a method of treating a cancer that expresses a mutant form of a RAS protein or a cancer associated with a RAS mutation, the additional therapeutic agent is an EGFR (HER1) inhibitor such as, e.g., cetuximab, panitumumab, vectibix, gefitinib, erlotinib, and Zactima.

In some embodiments of a method of treating a cancer that expresses a mutant form of a RAS protein or a cancer associated with a RAS mutation, the additional therapeutic agent is a HER2 inhibitors such as, e.g., lapatinib, tratuzumab, and pertuzumab.

In some embodiments of a method of treating a cancer that expresses a mutant form of a RAS protein or a cancer associated with a RAS mutation, the additional therapeutic agent is an mTOR inhibitors such as, e.g., temsirolimus, sirolimus/Rapamycin, and everolimus.

In some embodiments of a method of treating a cancer that expresses a mutant form of a RAS protein or a cancer associated with a RAS mutation, the additional therapeutic agent is a c-Met inhibitor.

In some embodiments of a method of treating a cancer that expresses a mutant form of a RAS protein or a cancer associated with a RAS mutation, the additional therapeutic agent is PI3K inhibitor such as, e.g., alpelisib, copanlisib, duvelisib, idelalisib, and umbralisib.

In some embodiments of a method of treating a cancer that expresses a mutant form of a RAS protein or a cancer associated with a RAS mutation, the additional therapeutic agent is an AKT inhibitors.

In some embodiments of a method of treating a cancer that expresses a mutant form of a RAS protein or a cancer associated with a RAS mutation, the additional therapeutic agent is a CDK inhibitor such as roscovitine and flavopiridol.

In some embodiments of a method of treating a cancer that expresses a mutant form of a RAS protein or a cancer associated with a RAS mutation, the additional therapeutic agent is a spindle assembly checkpoints inhibitors and targeted anti-mitotic agents such as PLK inhibitors, Aurora inhibitors (e.g. Hesperadin), checkpoint kinase inhibitors, and KSP inhibitors.

In some embodiments of a method of treating a cancer that expresses a mutant form of a RAS protein or a cancer associated with a RAS mutation, the additional therapeutic agent is a HDAC inhibitor such as, e.g., panobinostat, vorinostat, MS275, belinostat, and LBH589.

In some embodiments of a method of treating a cancer that expresses a mutant form of a RAS protein or a cancer associated with a RAS mutation, the additional therapeutic agent is HSP90 inhibitor.

In some embodiments of a method of treating a cancer that expresses a mutant form of a RAS protein or a cancer associated with a RAS mutation, the additional therapeutic agent is a HSP70 inhibitor.

In some embodiments of a method of treating a cancer that expresses a mutant form of a RAS protein or a cancer associated with a RAS mutation, the additional therapeutic agent is a proteasome inhibitor such as bortezomib and carfilzomib.

In some embodiments of a method of treating a cancer that expresses a mutant form of a RAS protein or a cancer associated with a RAS mutation, the additional therapeutic agent is s serine/threonine kinase inhibitors including MEK inhibitors (such as e.g. RDEA 119) and Raf inhibitors such as sorafenib.

In some embodiments of a method of treating a cancer that expresses a mutant form of a RAS protein or a cancer associated with a RAS mutation, the additional therapeutic agent is a farnesyl transferase inhibitor such as, e.g., tipifarnib.

In some embodiments of a method of treating a cancer that expresses a mutant form of a RAS protein or a cancer associated with a RAS mutation, the additional therapeutic agent is a tyrosine kinase inhibitor including, e.g., dasatinib, nilotibib, DAST, bosutinib, sorafenib, bevacizumab, sunitinib, AZD2171, axitinib, aflibercept, telatinib, imatinib mesylate, brivanib alaninate, pazopanib, ranibizumab, vatalanib, cetuximab, panitumumab, vectibix, gefitinib, erlotinib, lapatinib, tratuzumab, pertuzumab, and c-Kit inhibitors, and masitinib.

In some embodiments of a method of treating a cancer that expresses a mutant form of a RAS protein or a cancer associated with a RAS mutation, the additional therapeutic agent is a vitamin D receptor agonist.

In some embodiments of a method of treating a cancer that expresses a mutant form of a RAS protein or a cancer associated with a RAS mutation, the additional therapeutic agent is a Bcl-2 protein inhibitors such as ABT-737, BDA-366, gambogic Acid, HA14-1, navitoclax, obatoclax mesylate, S55746, sabutoclax, TW-37, and venetoclax. In some embodiments of a method of treating a cancer that expresses a mutant form of a RAS protein or a cancer associated with a RAS mutation, the additional therapeutic agent is venetoclax.

In some embodiments of a method of treating a cancer that expresses a mutant form of a RAS protein or a cancer associated with a RAS mutation, the additional therapeutic agent is a cluster of differentiation 20 receptor antagonists such as, e.g., rituximab.

In some embodiments of a method of treating a cancer that expresses a mutant form of a RAS protein or a cancer associated with a RAS mutation, the additional therapeutic agent is a ribonucleotide reductase inhibitors such as, e.g., gemcitabine.

In some embodiments of a method of treating a cancer that expresses a mutant form of a RAS protein or a cancer associated with a RAS mutation, the additional therapeutic agent is a tumor necrosis apoptosis inducing ligand receptor 1 agonists such as, e.g., mapatumumab.

In some embodiments of a method of treating a cancer that expresses a mutant form of a RAS protein or a cancer associated with a RAS mutation, the additional therapeutic agent is a 5-hydroxytryptamine receptor antagonists such as, e.g., rEV598, xaliprode, palonosetron hydrochloride, granisetron, Zindol, and AB-1001.

In some embodiments of a method of treating a cancer that expresses a mutant form of a RAS protein or a cancer associated with a RAS mutation, the additional therapeutic agent is an integrin inhibitors including alpha5-beta1 integrin inhibitors such as, e.g., E7820, JSM 6425, volociximab, and endostatin.

In some embodiments of a method of treating a cancer that expresses a mutant form of a RAS protein or a cancer associated with a RAS mutation, the additional therapeutic agent is an androgen receptor antagonists including, e.g., nandrolone decanoate, fluoxymesterone, android, prost-aid, andromustine, bicalutamide, flutamide, apo-cyproterone, apo-flutamide, chlormadinone acetate, Androcur, Tabi, cyproterone acetate, nilutamide, apalutamide, and enzalutamide.

In some embodiments of a method of treating a cancer that expresses a mutant form of a RAS protein or a cancer associated with a RAS mutation, the additional therapeutic agent is an aromatase inhibitors such as, e.g., anastrozole, letrozole, testolactone, exemestane, amino-glutethimide, and formestane.

In some embodiments of a method of treating a cancer that expresses a mutant form of a RAS protein or a cancer associated with a RAS mutation, the additional therapeutic agent is a matrix metalloproteinase inhibitors.

In some embodiments of a method of treating a cancer that expresses a mutant form of a RAS protein or a cancer associated with a RAS mutation, the additional therapeutic agent is a alitretinoin, ampligen, atrasentan bexarotene, bortezomib, bosentan, calcitriol, exisulind, fotemustine, ibandronic acid, miltefosine, mitoxantrone, I-asparaginase, procarbazine, dacarbazine, hydroxycarbamide, pegaspargase, pentostatin, tazaroten, velcade, gallium nitrate, canfosfamide, darinaparsin, and tretinoin.

### EXAMPLES

### Reagents and Cell Culture

Mouse embryonic fibroblasts (DU1473) null for both Hras and Nras are provided by M. Barbacid's laboratory (CNIO, Madrid, Spain). Cells are treated with 600 nM 4-hydroxy tamoxifen (Sigma-Aldrich. St. Louis, MO) for 11 d to eliminate the endogenous floxed Kras gene. The MEF panel was developed with cells lacking all endogenous Kras and were growth-arrested, transduced with lentiviral constructs expressing the WT RAS, mutant KRAS, or mutant BRAF allele. The peripheral blood mononuclearcells are purified from human blood samples (buffy coats). MRC5 and LS513 cells are obtained from the American Type Culture Collection (ATCC, Manassas, VA). Colo320 and SW620 are obtained from the DSMZ-German Collection of Microorganisms and CellCultures (Braunschweig, Germany) and the European Collection of Authenticated Cell Cultures (Salisbury, UK), respectively.

### Example 1: ATP-Based Cell Viability Assay

Cell viability is measured by the CellTiter-Glo assay (Promega, Fitchburg, WI), following the provided protocol. Briefly, for the primary screen, assay-ready plates containing 150 nl or 25 nl of 10 mM compounds are prepared with an Echo acoustic dispenser. The final concentration is 5 μM. Cells are then dispensed in the assay plates with a Multidrop Combi Reagent dispenser (ThermoFisher).

For dose-response curve experiments, serial dilutions are made with an automatic multi-channel pipetting robot, diluted on-line with the MultidropCombi Reagent dispenser, and added to the assay plates with a V-prep station. 72h (MEFs) or 96h (colorectal cancer cell lines) later, CellTiter-Glo reagent are added to the cells. After a 15 minincubation, luminescent signal is read with either the EnVision or PHERAstar plate reader.

### Example 2: Cellular Toxicity Assay

Cell toxicity is assessed with the 4-Plex Apoptosis Kit, which measures four parameters: cell viability (membrane integrity), caspase activity (caspase 3/7 substrate), annexin V binding (surface detection of phosphatidylserine), and mitochondrial damage (mitochondrial depolarization). One thousand cells are plated per well of a 384-well plate and are incubated with the tested compounds. Forty-eight hours later, 10 μl staining cocktail is added to the cells, and incubated for 1 h at room temperature (RT). Data are then acquired using the iQue Screener.

### Example 3: Biophysical Assays

Surface plasmon resonance (SPR) is performed with MASS2 using KRAS^{WT}1-169 and KRAS^{G12D} 1-169 as ligands, and the following experimental conditions: 50 mM HEPES, 150 mM NaCl, 1 mM MgCl₂, pH 7.4, 0.03% P20, and 5 μM GppNHp for GppNHp-loaded forms or 10 μM GDP for GDP-loaded forms; for the streptavidin chip: immo=3800-2500 RU, and theoretical Rₘₐₓ=50 RU.

### Example 4

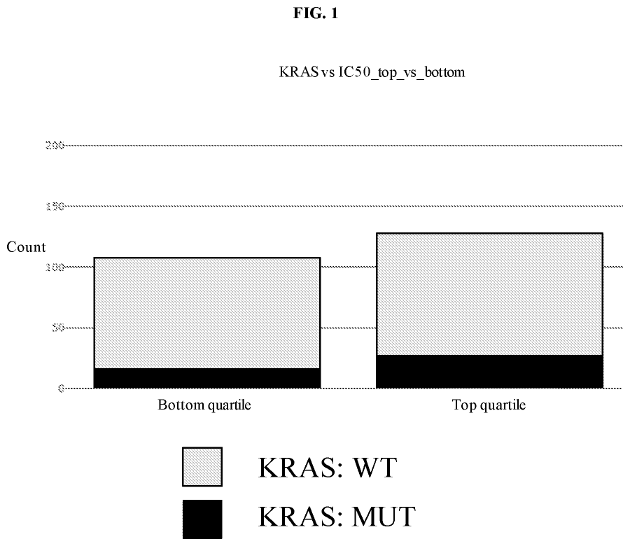

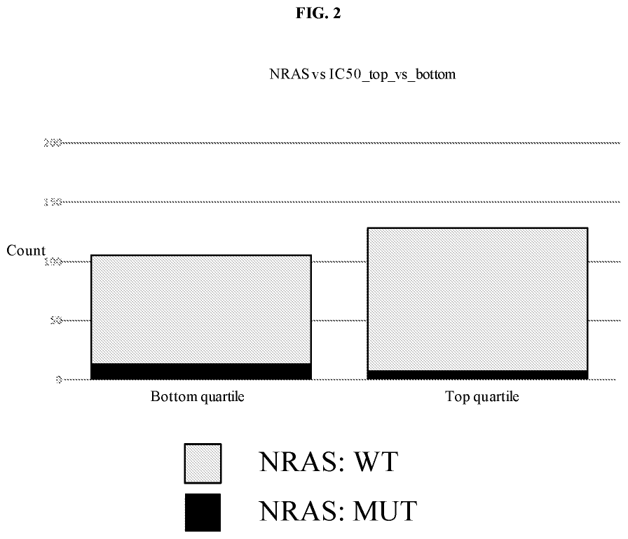

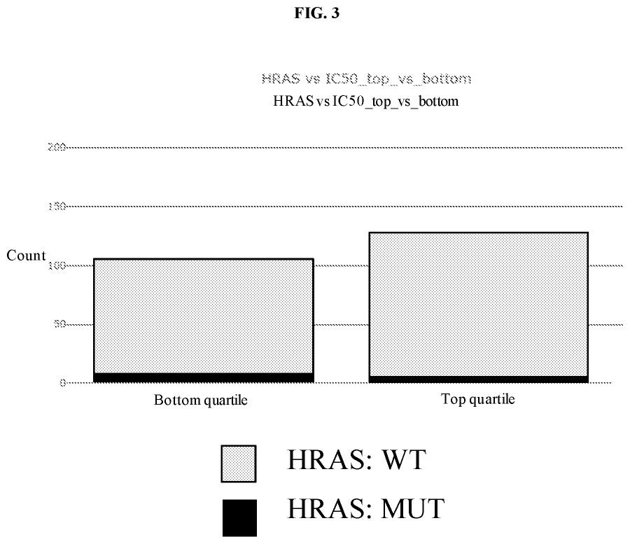

The antiproliferative activity of compound 1′ was evaluated against a panel of 500 tumor cell lines using 9 concentrations ranging from 3.16 nM to 31.6 μM and determining IC₅₀ for each cell line after 72 hours of treatment (OmniScreen™ which is a cell based screening service offered by the CRO Crown Bioscience, Inc.). The IC₅₀ range was 28 nM to 2.099 μM with a mean IC₅₀ of 113 nM. To determine associations of genomic alterations with compound 1′ sensitivity, 458 were mapped to a cell line present in DepMap (21Q3). Of the 458 cell lines mapped, there were 440 with available mutation data in DepMap. Cell line mutation and copy number data were accessed and for mutations, the data were filtered to include variants with a “damaging” or “other non-conserving” annotation only, in order to select variants which are more likely to be functionally important. The top quartile of cell lines based on their compound 1′IC50 values were defined as groups for comparison with the bottom quartile. IC50: top quartile (≥0.1520 μM; N=130) vs bottom quartile (≤0.0905 μM; N=110) are defined and Fisher's exact tests were performed to determine whether mutation status was associated with top/bottom quartile response groups. The results are displayed in FIG. 1, FIG. 2, and FIG. 3. This data demonstrates that cancer cell lines that harbor (H/K/N)RAS gene mutations are sensitive to CDK9 inhibition. Furthermore, (H/K/N)RAS gene mutations does not confer resistance CDK9 inhibitors. Exemplary antiproliferative activity of compound 1′ is shown in table 1.

TABLE 1

| | | Compound 1′ Absolute |
|---|---|---|
| Cell lines | Tissue Origin | IC₅₀(μM) |
| Panc 05.04 | Pancreas | 0.038 |
| Calu-6 | Lung | 0.039 |
| NCI-H358 | Lung | 0.041 |
| LCLC-97TM1 | Lung | 0.046 |
| LS513 | Large intestine/Colorectum | 0.051 |
| HEC-1-B | Uterus | 0.067 |
| UM-UC-3 | Bladder | 0.067 |
| NCI-H2122 | Lung | 0.07 |
| CoC1/DDP | Ovary | 0.072 |
| RBE | Liver/Bile duct | 0.072 |
| 143B | Bone | 0.073 |
| SU-DHL-10 | Lymphoma | 0.074 |
| NCI-H1944 | Lung | 0.075 |
| MOLP8 | Blood/Myeloma | 0.08 |
| SU.86.86 | Pancreas | 0.082 |
| HCC44 | Lung | 0.09 |
| DV-90 | Lung | 0.09 |

### Example 5: In Vitro Cytotoxicity

Cultivation of cells was performed according to standard procedures with the media recommended by the provider. The cells, in a total volume of 100 μL, were seeded in a 96-well plate with a white bottom (#3610). After a 24h incubation period at 37° C. and 5% CO₂ the medium was exchanged by adding 90 μL fresh medium. The treatment started by adding the test compound in 10 μL of culture medium to the cells in triplicates. Concentrations ranging from 10⁻⁶ M to 10⁻¹³ M were chosen. The proliferation was detected using the MTT assay (ATCC). At the end of the incubation period the MTT reagent was added to all samples for 4h. Lysis of the cells followed by addition of the detergent was done overnight. The formed dye was detected at 570 nm. The proliferation of cells was defined as the 100% value. The dose response curves allowed the determination of the respective IC₅₀ values, which are summarized in Table 2.

TABLE 2

| Cell Line | Indication | KRAS Mutation | Compound 1′ IC₅₀ (nM) |
|---|---|---|---|
| HCT116 | Colorectal | p.G13D | 46 |
| HCT15 | Colorectal | p.G13D | 33.5 |
| HCT8 | Colorectal | WT | 83 |
| HT-29 | Colorectal | WT | 83 |
| SW480 | Colorectal | p.G12V | 143 |
| SW620 | Colorectal | p.G12V | >500 |
| SW837 | Colorectal | pG12C | 620 |
| COLO205 | Colorectal | WT | 70 |
| A549 | Lung | p.G12S | 98 |
| NCI-H460 | Lung | p.Q61H | 75 |
| MIAPaCa2 | Pancreatic | pG12C | 128 |

The selection of cell lines with various KRAS mutations demonstrates that compound 1′ is active against multiple KRAS mutant colorectal and lung cancer cell lines.

What is claimed is:

1. A method of treating a cancer that expresses a mutant form of a RAS protein in a subject in need thereof, the method comprising administering to a subject in need a therapeutically-effective amount of a selective CDK9 inhibitor.

2. The method of claim 1, wherein the RAS protein is a mutant form of a NRAS protein.

3. The method of claim 1, wherein the RAS protein is a mutant form of a HRAS protein.

4. The method of claim 1, wherein the RAS protein is a mutant form of a KRAS protein.

5. The method of any one of claims 1-4, wherein the mutant form of the RAS protein comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

6. The method of any one of claims 1-5, wherein the mutant form of the RAS protein comprises one or more amino acid substitutions selected from the group consisting of G12C, G12D, G12V, G13S, and Q61R.

7. The method of any one of claims 1-6, wherein the mutant form of the RAS protein comprises the amino acid substitution G12C.

8. The method of any one of claims 1-6, wherein the mutant form of the RAS protein comprises the amino acid substitution G12D.

9. The method of any one of claims 1-6, wherein the mutant form of the RAS protein comprises the amino acid substitution G12V.

10. The method of any one of claims 1-6, wherein the mutant form of the RAS protein comprises the amino acid substitution G13S.

11. The method of any one of claims 1-6, wherein the mutant form of the RAS protein comprises the amino acid substitution Q61R.

12. The method of any one of claims 1-11, wherein the mutant form of the RAS protein comprises a secondary adaptive RAS mutation.

13. A method of treating a cancer associated with a RAS mutation in a subject in need thereof, the method comprising administering to a subject in need a therapeutically-effective amount of a selective CDK9 inhibitor.

14. The method of claim 13, wherein the RAS mutation is a NRAS mutation.

15. The method of claim 13, wherein the RAS mutation is a HRAS mutation.

16. The method of claim 13, wherein the RAS mutation is a KRAS mutation.

17. The method of any one of claims 13-16, wherein the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

18. The method of any one of claims 13-17, wherein the mutation comprises one or more amino acid substitutions selected from the group consisting of G12C, G12D, G12V, G13S, and Q61R.

19. The method of any one of claims 13-18, wherein the mutation comprises the amino acid substitution G12C.

20. The method of any one of claims 13-18, wherein the mutation comprises the amino acid substitution G12D.

21. The method of any one of claims 13-18, wherein the mutation comprises the amino acid substitution G12V.

22. The method of any one of claims 13-18, wherein the mutation comprises the amino acid substitution G13S.

23. The method of any one of claims 13-18, wherein the mutation comprises the amino acid substitution Q61R.

24. The method of any one of claims 13-23, wherein the mutation comprises secondary adaptive RAS mutation.

25. A method for treating cancer that expresses a mutant form of a RAS protein in a subject in need thereof with a selective CDK9 inhibitor, the method comprising:
(a) receiving a biological sample from the subject;
(b) assaying the biological sample for one or more RAS mutations;
(c) determining if one or more RAS mutations are present in the biological sample;
(d) employing the determination of one or more RAS mutations in the biological sample to predict responsiveness of the subject in need thereof to the compound, wherein the presence of one or more RAS mutations in the biological sample predicts that the subject will be responsive to the selective CDK9 inhibitor; and
(e) administering a therapeutically-effective amount of the selective CDK9 inhibitor to the subject.

26. A method of selecting a subject with cancer for treatment with a selective CDK9 inhibitor, the method comprising:
(a) receiving a biological sample from the subject with cancer; and
(b) determining if one or more RAS mutations are present in the biological sample.

27. A method for selecting a subject with cancer for treatment with a selective CDK9 inhibitor, the method comprising:
(a) receiving a biological sample from the subject;
(b) assaying the biological sample for one or more RAS mutations;
(c) determining if one or more RAS mutations are present in the biological sample;
(d) employing the determination of one or more RAS mutations in the biological sample to predict responsiveness of the subject in need thereof to the compound, wherein the presence of one or more RAS mutations in the biological sample predicts that the subject will be responsive to the selective CDK9 inhibitor; and
(e) administering a therapeutically-effective amount of the selective CDK9 inhibitor to the subject

28. A method of predicting a therapeutic response for the treatment of cancer to a selective CDK9 inhibitor in a subject with cancer, the method comprising:
(a) determining if one or more RAS mutations are present in a biological sample obtained from the subject with cancer; and
(b) determining whether the subject is likely to respond administration of the selective CDK9 inhibitor.

29. A method of contacting a cell with a selective CDK9 inhibitor, the method comprising:
(a) receiving a cell;
(b) assaying the cell for one or more RAS mutations;
(c) determining if one or more RAS mutations are present in the cell;
(d) employing the determination of one or more RAS mutations in the cell to predict responsiveness of the cell to the selective CDK9 inhibitor, wherein the presence of one or more RAS mutations in the cell predicts that the cell will be responsive to the selective CDK9 inhibitor; and
(e) selectively contacting a cell predicted to be responsive to the selective CDK9 inhibitor with the selective CDK9 inhibitor.

30. The method of any one of claims 25-29, wherein the biological sample is a tumor biopsy.

31. The method of any one of claims 25-29, wherein the biological sample is an aspirate.

32. The method of any one of claims 25-31, wherein the RAS mutation is a NRAS mutation.

33. The method of any one of claims 25-31, wherein the RAS mutation is a HRAS mutation.

34. The method of any one of claims 25-31, wherein the RAS mutation is a KRAS mutation.

35. The method of any one of claims 25-34, wherein the mutation comprises one or more amino acid substitutions selected from the group consisting of G12S, G12C, G12R, G12D, G12V, G12F, G12L, G12N, G13C, G13R, G13S, G13A, G13V, G13P, G13D, S17G, P34S, A59E, A59G, A59T, Q61K, Q61L, Q61R, Q61H, K117N, A146P, A146T, A146V, and D153V.

36. The method of any one of claims 25-35, wherein the mutation comprises one or more amino acid substitutions selected from the group consisting of G12C, G12D, G12V, G13S, and Q61R.

37. The method of any one of claims 25-36, wherein the mutation comprises the amino acid substitution G12C.

38. The method of any one of claims 25-36, wherein the mutation comprises the amino acid substitution G12D.

39. The method of any one of claims 25-36, wherein the mutation comprises the amino acid substitution G12V.

40. The method of any one of claims 25-36, wherein the mutation comprises the amino acid substitution G13S.

41. The method of any one of claims 25-36, wherein the mutation comprises the amino acid substitution Q61R.

42. The method of any one of claims 25-41, wherein the mutation comprises a secondary adaptive RAS mutation.

43. The method of any one of claims 1-42, wherein the cancer is bladder cancer, brain cancer, brain metastases, breast cancer, cervical cancer, colorectal cancer, endometrial cancer, gastrointestinal cancer, glioblastoma, head and neck cancer, leukemias, liver cancer, lung cancer, lymphomas, melanoma, multiple myeloma, ovarian cancer, pancreatic cancer, peritoneal cancer, prostate cancer, renal cancer, salivary gland carcinoma, thyroid cancer, or uterine carcinoma.

44. The method of any one of claims 1-42, wherein the cancer is non-small cell lung cancer, pancreatic cancer, or glioblastoma.

45. The method of any one of claims 1-42, wherein the cancer is colorectal cancer, non-small cell lung cancer, or pancreatic cancer.

46. The method of any one of claims 1-42, wherein the cancer is glioblastoma.

47. The method of any one of claims 1-42, wherein the cancer is colorectal cancer.

48. The method of any one of claims 1-42, wherein the cancer is non-small cell lung cancer.

49. The method of any one of claims 1-42, wherein the cancer is pancreatic cancer.

50. The method of any one of claims 1-49, wherein the selective CDK9 inhibitor is administered in multiple 21-day cycles.

51. The method of any one of claims 1-49, wherein the selective CDK9 inhibitor is administered in at least one 21-day cycle.

52. The method of any one of claims 1-49, wherein the selective CDK9 inhibitor is administered in at least two 21-day cycles.

53. The method of any one of claims 1-49, wherein the selective CDK9 inhibitor is administered in at least three 21-day cycles.

54. The method of any one of claims 1-49, wherein the selective CDK9 inhibitor is administered in at least four 21-day cycles.

55. The method of any one of claims 1-49, wherein the selective CDK9 inhibitor is administered in at least five 21-day cycles.

56. The method of any one of claims 1-49, wherein the compound is administered in at least six 21-day cycles.

57. The method of any one of claims 1-49, wherein the selective CDK9 inhibitor is administered in at least 12 21-day cycles.

58. The method of any one of claims 1-57, wherein the selective CDK9 inhibitor is administered once weekly.

59. The method of any one of claims 1-57, wherein the selective CDK9 inhibitor is administered twice weekly.

60. The method of any one of claims 1-57, wherein the selective CDK9 inhibitor is administered weekly three days on and four days off.

61. The method of any one of claims 1-60, wherein the method further comprises administering an additional therapeutic agent.

62. The method of claim 61, wherein the additional therapeutic agent is an AKT inhibitor, BCL2 inhibitor, BTK inhibitor, EGFR inhibitor, farnesyl transferase inhibitor, HER2 inhibitor, MEK inhibitor, mTOR inhibitor, PI3K inhibitor, RAF inhibitor, RAS inhibitor, or any combinations thereof.

63. The method of claim 61 or 62, wherein the selective CDK9 inhibitor and the additional therapeutic agent are administered sequentially.

64. The method of claim 61 or 62, wherein the selective CDK9 inhibitor and the additional therapeutic agent are administered simultaneously.

65. The method of claim 61 or 62, wherein the selective CDK9 inhibitor is administered before the additional therapeutic agent.

66. The method of claim 61 or 62, wherein the selective CDK9 inhibitor is administered after the additional therapeutic agent.

67. The method of any one of claims 1-66, wherein the selective CDK9 inhibitor is a compound that is 5-fluoro-4-(4-fluoro-2-methoxyphenyl)-N-{4-[(S-methylsulfonimidoyl)methyl]pyridin-2-yl}pyridin-2-amine: 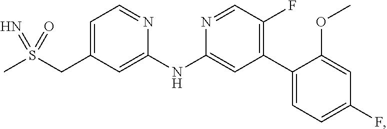 [SMILES: COc1cc(F)ccc1-c1cc(Nc2cc(CS(C)(=N)=O)ccn2)ncc1F]
or an enantiomer thereof, or a pharmaceutically acceptable salt or solvate thereof.

68. The method of claim 67, wherein the enantiomer of the compound is the (+) 5-fluoro-4-(4-fluoro-2-methoxyphenyl)-N-{4-[(S-methylsulfonimidoyl)methyl]pyridin-2-yl}pyridin-2-amine.

69. The method of claim 67 or 68, wherein the compound is administered at a dose of between about 1 mg and about 50 mg.

70. The method of claim 67 or 68, wherein the compound is administered at a dose of between about 1 mg and about 30 mg.

71. The method of claim 67 or 68, wherein the compound is administered at a dose of between about 10 mg and about 30 mg.

72. The method of claim 67 or 68, wherein the compound is administered at a dose of between about 20 mg and about 30 mg.

73. The method of claim 67 or 68, wherein the compound is administered at a dose of about 5 mg.

74. The method of claim 67 or 68, wherein the compound is administered at a dose of about 10 mg.

75. The method of claim 67 or 68, wherein the compound is administered at a dose of about 15 mg.

76. The method of claim 67 or 68, wherein the compound is administered at a dose of about 20 mg.

77. The method of claim 67 or 68, wherein the compound is administered at a dose of about 25 mg.

78. The method of claim 67 or 68, wherein the compound is administered at a dose of about 30 mg.

79. The method of any one of claims 1-66, wherein the selective CDK9 inhibitor is KB-0742, PRT2527, A-1592668, AZD4573, or fadraciclib.

80. The method of any one of claims 1-66, wherein the selective CDK9 inhibitor is KB-0742.

81. The method of any one of claims 1-66, wherein the selective CDK9 inhibitor is PRT2527.

82. The method of any one of claims 1-66, wherein the selective CDK9 inhibitor is A-1592668.

83. The method of any one of claims 1-66, wherein the selective CDK9 inhibitor is AZD4573.

84. The method of any one of claims 1-66, wherein the selective CDK9 inhibitor is fadraciclib.
